(19) United States
(12) Patent Application Publication
(10) Pub. No.: US 2025/0009647 A1
(43) Pub. Date: Jan. 9, 2025

(54) PHARMACEUTICAL COMPOSITION

(71) Applicant: MedinCell S.A., Jacou (FR)

(72) Inventors: Juliette SERINDOUX, Jacou (FR); Adolfo LÓPEZ-NORIEGA, Jacou (FR)

(73) Assignee: MedinCell S.A., Jacou (FR)

(21) Appl. No.: 18/294,961

(22) PCT Filed: Aug. 5, 2022
(86) PCT No.: PCT/EP2022/072148

§ 371 Date: Feb. 2, 2024

(30) Foreign Application Priority Data
Aug. 5, 2021 (GB) 2111321.2

(51) Int. Cl. A61K 9/00; A61K 31/343; A61K 31/40; A61K 38/12; A61K 47/10; A61K 47/12; A61K 47/20; A61K 47/34

(52) U.S. Cl. CPC A61K 9/0024; A61K 31/343; A61K 31/40; A61K 38/12; A61K 47/10; A61K 47/12; A61K 47/20; A61K 47/34

(57) ABSTRACT

The present invention provides a pharmaceutical composition comprising or consisting of at least one polyether-polyester copolymer, wherein the copolymer has the formula: B(A)n wherein B represents a polyether and comprises polyethylene glycol (PEG), each A represents a polyester arm and n is an integer from 1 to 8; at least one nucleophilic compound; at least one organic solvent; and up to 10% (w/w) of at least one acidic compound having a pKₐ(H₂O) of less than 3.

### FIELD OF THE INVENTION

The present invention relates to pharmaceutical compositions with improved stability which are suitable for sustained release of an active pharmaceutical ingredient. The pharmaceutical compositions are suitable for parenteral use and may be used for any indication or dosage regimen where a sustained release is desired.

### BACKGROUND OF THE INVENTION

Different types of sustained release formulations are in use today. WO1993/24150 and WO2003/000778, disclose the formation of salts with a charged drug substance and a modified copolymer wherein the used block (co-)polymers are chemically modified to exhibit negative charges at the end of their PLA chains. WO2007/084460 describes an injectable polymeric composition with extended stability used for the delivery of peptides. The peptide active forms a salt with a strong acid. The disclosed polymers do not comprise PEG.

WO2016/061296 describes a pharmaceutical composition which is an injectable biodegradable polymeric formulation, which may be a PLA-based polymer, linear or branched, with a nucleophilic bioactive substance in an organic solvent.

U.S. Pat. No. 8,173,148 describes a composition comprising a biodegradable biocompatible polyester (linear or branched), a nucleophilic bioactive agent having at least one nitrogen group in a free base or salt form and a stabilizing associate which is a polycarbocylic acid. In the compositions of U.S. Pat. No. 8,173,148 the acidic compound is mixed with the nucleophilic bioactive agent prior to the nucleophile contact with the polyester to be effective.

WO2005007122A2 and family member U.S. Pat. No. 8,343,513 disclose a sustained release formulations comprising a biocompatible and biodegradable polymer, at least one nucleophilic substance capable of catalysing ester bond cleavage and causing molecular weight reduction of the polymer, and an amount of an acid additive such that the polymer in the formulation is less susceptible to molecular weight reduction as compared to the formulation without the acid additive. The acid additive may have a pKa of less than 5.00: however all of the specific acid compounds disclosed have a pKa of greater than 3. The low pKₐ acids are used to extend drug product stability. The compositions typically comprise PLA or PLGA based (co)polymers, including PEG-PLGA and PEG-PLA, but multi-arm copolymers or combinations of PEG-polyester copolymers are not disclosed. In addition, the exemplified compositions comprise microparticles and do not typically comprise solvent in the final products.

Although the above-mentioned documents describe the stabilization of pharmaceutical formulations, there is still a need to provide formulations with improved stability.

### SUMMARY OF THE INVENTION

The present invention relates to pharmaceutical compositions with improved stability properties, in particular liquid pharmaceutical compositions with improved stability properties, suitable for generating an in situ depot when injected into an aqueous environment.

An aspect according to the invention provides a pharmaceutical composition comprising or consisting of
a) at least one polyether-polyester copolymer, wherein the copolymer has the formula:

B(A)ₙ
wherein B represents a polyether and comprises polyethylene glycol (PEG), each A represents a polyester arm and n is an integer from 1 to 8;
b) at least one nucleophilic compound;
c) at least one organic solvent; and
d) up to 10% (w/w) of at least one acidic compound having a pKₐ(H₂O) of less than 3.

The inventors have surprisingly found that the above-mentioned pharmaceutical composition has improved stability, i.e. a reduction in degradation of the polyether-polyester copolymer over time. Without being bound by theory, the present inventors understand that the presence of a specific amount of an acid with a specific low pKₐ prevents nucleophile induced polyester degradation. This effect is achieved even without prior reaction of the acidic compound with the nucleophilic compound before addition to the at least one polyether-polyester copolymer, i.e. the stabilization effect does not rely on the prior formation of a salt or complex with the acid.

Preferred embodiments of the invention provide a pharmaceutical composition as defined above wherein the at least one polyether-polyester copolymer a) is selected from;
i. a multi-arm copolymer having 3 to 8 polyester arms attached to a central core which is a multi-arm polyether comprising PEG and wherein each polyether arm has from 2 to 150 ethylene oxide repeat units and each polyester arm has from 4 to 200 repeat units; and
ii. a triblock copolymer, wherein the triblock copolymer has the formula:

Av-Bw-Ax
wherein A is a polyester and B is PEG and v and x are the number of repeat units ranging from 1 to 3,000 and w is the number of repeat units ranging from 3 to 300 and v=x or v≠x; and
iii. a diblock copolymer, wherein the diblock copolymer has the formula:

Cy-Az
wherein A is a polyester and C is an end-capped PEG and y and z are the number of repeat units with y ranging from 2 to 250 and z ranging from 1 to 3,000;
iv. or any combination thereof.

Each acidic compound has a pKₐ(H₂O) of less than 3.00. Each acidic compound preferably has a pKₐ(H₂O) of from −15.00 to 2.97, more preferably from about −3.00 to about 2.90, optionally from about 0.50 to about 2.75, optionally from about 1.40 to about 2.75.

In a preferred embodiment, the composition is liquid at room temperature and forms a semi solid or solid implant when injected into an aqueous environment. The compositions of the invention form an “in situ depot” which is a semi-solid, localized mass formed by precipitation of the pharmaceutical composition after injection of the composition into the subject. The pharmaceutical composition comprises copolymers which are substantially insoluble in aqueous solution. Thus, when the pharmaceutical composition contacts the aqueous environment of the human or animal body, a phase inversion occurs causing the composition to change from a liquid to a semi-solid, i.e. precipitation of the composition occurs, leading to formation of an “in situ depot”.

The acidic compound may be an inorganic acid or a carboxylic acid, optionally a polycarboxylic acid, optionally a di or tricarboxylic acid.

In a preferred embodiment the acidic compound d) is selected from aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid, tartaric acid citraconic acid, methylphosphonic acid, ethylphosphonic acid, propylphosphonic acid, butylphosphonic acid, pentylphosphonic acid, hexylphosphonic acid, heptylphosphonic, octylphosphonic acid, nicotinic acid, hydroiodic acid, chromic acid, trifluoromethane sulfonic acid, trichloroacetic acid, dichloroacetic acid, bromoacetic acid, chloroacetic acid, cyanoacetic acid, 2-chloropropanoic acid, 2-chlorobutanoic acid, 4-cyanobutanoic acid, perchloric acid, a phosphoric acid or a combination thereof.

In particularly preferred embodiments of the invention the acidic compound is selected from aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid or tartaric acid or a combination thereof, preferably salicyclic acid, oxalic acid, malonic acid, sulfamic acid, pamoic acid or any combination thereof.

Typically the polyester of the polyether-polyester copolymer is poly(D,L-lactic acid) (PLA), poly(D,L-lactic-co-glycolic acid) (PLGA) or poly(ε-caprolactone-co-lactic acid) (PCLA).

The end-capped polyethylene glycol of the diblock copolymer is preferably methoxy-polyethylene glycol.

In a preferred embodiment the polyester is poly(D,L-lactic acid) (PLA).

In one embodiment the polyether-polyester copolymer is a multi-arm copolymer wherein each polyether arm has from 2 to 150 ethylene oxide repeat units and each polyester arm has from 4 to 200 repeat units.

Typically the polyether-polyester copolymer is a multi-arm copolymer having a molar ratio of the ester repeat unit to the ethylene oxide repeat unit of from 1 to 10, preferably from 2 to 6.

In a preferred embodiment the polyether-polyester copolymer is a multi-arm copolymer having from 3 to 8 arms.

If the polyether-polyester copolymer is a multi-arm copolymer the central core is a multi-arm polyether which may be obtainable from poly(ethylene glycol) (PEG) and a polyol. Preferably, the polyol comprises at least three hydroxyl groups, optionally wherein the polyol is a hydrocarbon substituted with at least three hydroxyl groups, optionally 3, 4, 5, 6 or 8 hydroxyl groups. Typically the polyol is pentaerythritol (PE), dipentaerythritol, trimethylolpropane (TMP), glycerol, erythritol, xylitol, di(trimethylolpropane (diTMP) sorbitol, or inositol.

In one embodiment the polyol further comprises one or more ether groups.

In some embodiment for the multi-arm copolymer, the number of arms is 4, the molecular weight of the PEG core is 2 kDa, and the lactic acid/ethylene oxide molar ratio is 3 or 4.

In a preferred embodiment the polyether-polyester copolymer is a mixture of a diblock copolymer and a triblock copolymer. In one embodiment the molar ratio of the ester repeat unit to the ethylene oxide repeat unit for the diblock copolymer is from 0.8 to 15, preferably from 1 to 10. In one embodiment the molar ratio of the ester repeat unit to the ethylene oxide repeat unit for the triblock copolymer is from 0.5 to 22, preferably from 0.5 to 10, most preferably from 1 to 6.

In some embodiment for the triblock copolymer the molecular weight of the PEG is 1 kDa, and the lactic acid/ethylene oxide molar ratio is 4 or 6 and for the diblock copolymer the molecular weight of the PEG is 2 kDa, and the lactic acid/ethylene oxide molar ratio is 3.

In some embodiment for the triblock copolymer the molecular weight of the PEG is 1 kDa, and the lactic acid/ethylene oxide molar ratio is 6 and for the diblock copolymer the molecular weight of the mPEG is 1 kDa, and the lactic acid/ethylene oxide molar ratio is 4.

In some embodiment for the triblock copolymer the molecular weight of the PEG is 2 kDa, and the lactic acid/ethylene oxide molar ratio is 2 and for the diblock copolymer the molecular weight of the mPEG is 2 kDa, and the lactic acid/ethylene oxide molar ratio is 3.

Typically the nucleophilic compound comprises one or more functional groups selected from —SH, —OH, a primary amine, a secondary amine, a tertiary amine, a heterocyclic group and combinations thereof.

In one embodiment the nucleophilic compound is an active pharmaceutical ingredient.

In one embodiment the active pharmaceutical ingredient is a free base or is a salt of an acid having a pKa(H₂O) of greater than 3. In one embodiment, the active pharmaceutical ingredient is octreotide acetate, liothyronine, escitalopram free base, atorvastatin calcium trihydrate or combination thereof.

In another embodiment the nucleophilic compound is not an active pharmaceutical ingredient and the composition further comprises at least one active pharmaceutical ingredient.

In one embodiment the nucleophilic compound is an alcohol, optionally a C₁ to C₈ alcohol, optionally glycerol, sorbitol, methanol, ethanol, propanediol, propylene glycol, polyethylene glycol, preferably methanol, propylene glycol, polyethylene glycol or derivatives or mixtures thereof.

In one embodiment the nucleophilic compound is a saccharide, disaccharide or polysaccharide, optionally sucrose, dextrose, cyclodextrin, chitosan or mixtures thereof.

In one embodiment the nucleophilic compound is an amino acid, peptide, or polypeptide, optionally lysine, arginine, histidine or serine.

In one embodiment the nucleophilic compound is water.

In one embodiment the nucleophilic compound is a further organic solvent, i.e. a solvent in addition to the at least one organic solvent defined in c) above, optionally pyrrolidone-2, glycofurol, pyridine, nitromethane, triethylamine, N,N-dimethylaniline, N,N-diemthyldecanamide, N,N-dimethyloctanamide, 2,4,6-collidine or mixtures thereof.

In one embodiment the composition comprises at least one active pharmaceutical ingredient and the nucleophilic compound is a solubility enhancer, a porogen or a phase exchange modifier.

A solubility enhancer can be a further organic solvent selected from the group consisting of benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, triacetin, tributyrin, tripropionin, glycofurol, pyridine, nitromethane, trimethylamine, N,N-dimethylaniline, N,N-dimethyldecanamide, N,N-dimethyloctanamide, 2,4,6-collidine and mixtures thereof.

The solubility enhancer may alternatively be a solid compound which is soluble in the at least one organic solvent c).

In one embodiment the solubility enhancer is selected from the list consisting of propylene glycol, polyethylene glycol, glycerol, sorbitol, a cyclodextrin and mixtures thereof.

In one embodiment the nucleophilic compound is a porogen or a phase exchange modifier.

Examples of porogens and/or phase exchange modifiers are saccharides, disaccharides or polysaccharides, such as sucrose or dextrose, or fatty acids, such as a triglyceride, or vegetable oil, or alcohol, such as a C₁ to C₈ alcohol or polyethylene glycol.

In one embodiment the porogen or the phase exchange modifier is selected from the list consisting of saccharides, polysaccharides or alcohols.

Typically the at least one organic solvent c) is selected from the group consisting of benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, triacetin, tributyrin, tripropionin, glycofurol or a mixture thereof, preferably DMSO, NMP and mixtures thereof.

In a preferred embodiment the acidic compound has a pKₐ(DMSO) lower than 10, preferably lower than 8.

In some embodiments the amount of the at least one acidic compound is from 0.005% (w/w) to 10% (w/w), optionally 0.55% (w/w) to 10% (w/w), or 0.005% (w/w) to 0.45% (w/w), preferably 0.01% (w/w) to 4.0% (w/w) of the total composition.

The molar amount of the acidic compound may be 0.05% to 300% relative to the molar amount of the nucleophilic compound, preferably 0.1% to 250%. In one embodiment the nucleophilic compound contains at least one —OH group and the molar amount of the acidic compound is equal to or lower than 100% relative to the molar amount of the nucleophilic amount, preferably 0.05% to 100% relative to the molar amount of the nucleophilic compound. In one embodiment the nucleophilic compound contains at least one nitrogen containing reactive group such as a primary amine or a secondary amine, and the molar amount of the acidic compound is equal to or greater than 100% relative to the molar amount of the nucleophilic compound, preferably 100% to 300% relative to the molar amount of the nucleophilic compound.

In preferred embodiments the total amount of the polyether-polyester copolymer is 2% (w/w) to 80% (w/w), optionally 10 to 50% (w/w), optionally 20 to 40% (w/w) of the total composition.

In one embodiment the polyether-polyester copolymer is a multi-arm copolymer i) and the amount of the multi-arm copolymer is from 20 to 60% (w/w), optionally 20 to 50% (w/w) of the total composition.

When the composition comprises a diblock copolymer and a triblock copolymer, typically the amount of the diblock copolymer is from 2 to 30% (w/w), optionally 10 to 30% (w/w), optionally 10 to 20% (w/w) of the total composition; and the amount of the triblock copolymer is from 2 to 30% (w/w), optionally 10 to 30% (w/w), optionally 10 to 20% (w/w) of the total composition.

Typically the amount of the active pharmaceutical ingredient is 0.05% (w/w) to 60% (w/w), optionally 0.05 to 20% (w/w), optionally 0.05 to 10% (w/w), optionally 0.05 to 5% (w/w), optionally 0.05 to 2% (w/w) of the total composition.

Typically the amount of the organic solvent is at least 20% (w/w) of the total composition, optionally 20 to 80% (w/w), optionally 20 to 60% (w/w).

In preferred embodiments the composition is stable for at least 2 weeks storage at room temperature or 2 to 8° C., preferably at least 4 weeks storage at room temperature or 2 to 8° C.

In one embodiment the concentration of the active pharmaceutical ingredient in the composition reduces by less than 20%, preferably less than 10%, more preferably less than 5% after 2 weeks storage at room temperature or 2 to 8° C., preferably 4 weeks storage at room temperature or 2 to 8° C. relative to the initially formulated composition.

In one embodiment the dynamic viscosity of the composition reduces by less than 10%, preferably less than 5% after 2 weeks storage at room temperature or 2 to 8° C., preferably 4 weeks storage at room temperature or 2 to 8° C. relative to the initially formulated composition.

In a further aspect of the invention, provided is method for preparing a pharmaceutical composition as described above comprising or consisting of the steps of:
i. dissolving the at least one polyether-polyester copolymer a) as defined above in the at least one organic solvent c);
ii. adding to the product of step i) at least one acidic compound d) as defined above and at least one nucleophilic compound b) as defined above, optionally wherein the nucleophilic compound b) is an active pharmaceutical ingredient; and
iii. homogenizing the product of step ii), thereby obtaining the pharmaceutical composition.

In a preferred embodiment the at least one acidic compound and the at least one nucleophilic compound do not form a salt or complex prior to step ii). In an embodiment of the invention, the at least one acidic compound and the at least one nucleophilic compound are not contacted or mixed together prior to step ii). A great advantage of the present invention over prior art methods is that no initial step is required in which the acidic compound is reacted with the nucleophilic compound (which may be an API) before the nucleophilic compound is mixed with the other components of the composition, in particular the copolymer. In an embodiment of the present invention, all of the reactants can be mixed together in a single step, and the acid can achieve its stabilization effect without first having to be reacted with the nucleophilic compound.

In a preferred embodiment step ii) consists of mixing the components in a single step.

In another aspect, the invention provides a method for preparing a pharmaceutical composition as described above comprising or consisting of the steps of:
i. dissolving the at least one polyether-polyester copolymer a) as defined in any preceding claim in the at least one organic solvent c);
ii. adding to the product of step i) at least one acidic compound d) as defined above or at least one nucleophilic compound b) as defined above, and then homogenizing the product;
iii. if at least one acidic compound d) is added in step ii) then subsequently adding at least one nucleophilic compound b) as defined above; or if at least one nucleophilic compound b) is added in step ii) then subsequently adding at least one acidic compound d) as defined above; and
iv. homogenizing the product of step iii), thereby obtaining the pharmaceutical composition; optionally wherein the nucleophilic compound b) is an active pharmaceutical ingredient.

In embodiments of the invention, the nucleophilic compound is not an active pharmaceutical ingredient and an active pharmaceutical ingredient is added after step i).

In one embodiment the active pharmaceutical ingredient is previously dissolved in the organic solvent. In one embodiment the acidic compound is previously dissolved in the organic solvent. In one embodiment the nucleophilic compound is previously dissolved in the organic solvent. In one embodiment the pharmaceutical composition obtained in step iii. or iv. is filtered.

In a further aspect, provided is a pharmaceutical composition obtainable or obtained by the method defined above.

### DETAILED DESCRIPTION

An aspect according to the invention provides a pharmaceutical composition comprising or consisting of
a) at least one polyether-polyester copolymer, wherein the copolymer has the formula:

B(A)ₙ
wherein B represents a polyether and comprises polyethylene glycol (PEG), each A represents a polyester arm and n is an integer from 1 to 8;
b) at least one nucleophilic compound;
c) at least one organic solvent; and
d) up to 10% (w/w) of at least one acidic compound having a pKₐ(H₂O) of less than 3.

In a preferred embodiment, the composition is liquid at room temperature and forms a semi solid or solid implant when injected into an aqueous environment. The composition described above is typically suitable for forming a depot when injected into the body, i.e. an “in situ depot”.

The compositions of the invention are administered via depot injection. The term “depot injection” is an injection of a flowing pharmaceutical composition, usually subcutaneous, intradermal or intramuscular that deposits a drug in a localized mass, such as a solid or semi-solid mass, called a “depot”. The depots as defined herein are in situ forming upon injection. Thus, the formulations can be prepared as solutions or suspensions and can be injected into the body.

An “in situ depot” is a solid or semi-solid, localized mass formed by precipitation of the pharmaceutical composition after injection of the composition into the subject. The pharmaceutical composition comprises copolymers which are substantially insoluble in aqueous solution. Thus, when the pharmaceutical composition contacts the aqueous environment of the human or animal body, a phase inversion occurs causing the composition to change from a liquid to a solid, i.e. precipitation of the composition occurs, leading to formation of an “in situ depot”.

An “in situ depot” can be clearly distinguished from hydrogel pharmaceutical formulations described in the prior art. Hydrogels have three-dimensional networks that are able to absorb large quantities of water. The polymers making up hydrogels are soluble in aqueous solution. By contrast, the polymers used in the present invention are substantially insoluble in aqueous solution. The pharmaceutical compositions of the invention typically contain low concentrations of water, or water is absent. For example, the pharmaceutical compositions of the invention may comprise less than 0.5% (w/w) water.

In one embodiment, the pharmaceutical compositions of the invention comprise at least one polyether-polyester copolymer as defined above, at least one nucleophilic compound as defined above which may be an active pharmaceutical ingredient, at least one organic solvent as defined above and at least one acidic compound as defined above.

In another embodiment pharmaceutical compositions of the invention comprise at least one polyether-polyester copolymer as defined above, at least one nucleophilic compound as defined above, at least one active pharmaceutical ingredient, at least one organic solvent and at least one acidic compound as defined above.

Thus it can be seen that the nucleophilic compound can be an API, or the nucleophilic compound is not an API, and the API is provided as a separate compound.

The compositions of the invention comprise at least one polyether-polyester copolymer.

As mentioned above, B represents a polyether and comprises or is polyethylene glycol (PEG) or end-capped PEG. For the multi-arm copolymer i) this typically means that B is a multi-arm polyether obtainable from the reaction of PEG with a polyol, or more typically the reaction of the precursor of PEG which is ethylene oxide with a polyol. When the polyether-polyester copolymer is a triblock copolymer B is PEG. When the polyether-polyester copolymer is a diblock copolymer B is an end-capped PEG such as methoxy-PEG.

In preferred embodiments the pharmaceutical composition comprises at least one polyether-polyester copolymer a) which is selected from;
i. a multi-arm copolymer having 3 to 8 polyester arms attached to a central core which is a multi-arm polyether comprising PEG and wherein each polyether arm has from 2 to 150 ethylene oxide repeat units and each polyester arm has from 4 to 200 repeat units; and
ii. a triblock copolymer, wherein the triblock copolymer has the formula:

Av-Bw-Ax
wherein A is a polyester and B is PEG and v and x are the number of repeat units ranging from 1 to 3,000 and w is the number of repeat units ranging from 3 to 300 and v=x or v≠x; and
iii. a diblock copolymer, wherein the diblock copolymer has the formula:

Cy-Az
wherein A is a polyester and C is an end-capped PEG and y and z are the number of repeat units with y ranging from 2 to 250 and z ranging from 1 to 3,000;
iv. or any combination thereof.

The copolymers used in the present invention can be described as “bioresorbable” or “biodegradeable” which means that the block copolymers undergo hydrolysis in vivo to form their constituent (m)PEG and oligomers or monomers or repeat units derived from the polyester block. For example, poly(ε-caprolactone-co-lactic acid) (PCLA) undergoes hydrolysis to form 6-hydroxycaproic acid (6-hydroxyhexanoic acid) and lactic acid. The result of the hydrolysis process leads to a progressive mass loss of the depot and ultimately to its disappearance.

The molecular weight of each copolymer is the number average molecular weight. The number average molecular weight is typically measured using gel permeation chromatography (GPC) using a calibration curve obtained from polystyrene standards.

In a preferred embodiment of the invention, the polyether of the polyether-polyester copolymer comprises poly(ethylene glycol) (PEG) or is PEG, or end-capped PEG such as methoxy-PEG.

In one embodiment, the polyester of the polyether-polyester copolymer is poly(D,L-lactic acid) (PLA), poly(D,L-lactic-co-glycolic acid) (PLGA), or poly(ε-caprolactone-co-lactic acid) (PCLA), preferably poly(D,L-lactic acid). The polyesters are terminated by a hydroxyl (—OH) end group. The polymers according to the present invention preferably have an acid number below 15 or preferably below 5. Acid number is the measure of the amount of free acids in a substance usually expressed as the number of milligrams of potassium hydroxide (KOH) required to neutralize one gram of the substance.

The PEG-PLA copolymer is obtainable by reacting PEG with D,L-lactide, preferably by ring-opening polymerisation of the D,L-lactide initiated by the PEG. The polyether-PLGA copolymer is obtainable by reacting PEG with D,L-lactide and glycolide, preferably by ring-opening polymerisation of the D,L-lactide and the glycolide initiated by the PEG. The polyether-PCLA copolymer is obtainable by reacting PEG with ε-caprolactone and D,L-lactide, preferably by ring opening of ε-caprolactone and D,L-lactide initiated by the PEG.

The end-capped polyethylene glycol of the diblock copolymer is preferably methoxy-polyethylene glycol.

In one embodiment the polyether-polyester copolymer is a multi-arm copolymer. The term “multi-arm copolymer” means a polymer with at least three polyester arms attached to a central core, the central core of the invention comprising a polyether. The polyester arms may be referred to as “branches”, “arms” or “chains”. The term “multi-arm copolymer” has the same meaning as the term “star copolymer” or “star-shaped copolymer” or “multi-branched copolymer” and these terms are used interchangeably throughout.

In one embodiment the polyether-polyester copolymer is a multi-arm copolymer wherein each polyether arm has from 2 to 150 ethylene oxide repeat units and each polyester arm has from 4 to 200 repeat units.

Typically the polyether-polyester copolymer is a multi-arm copolymer having a molar ratio of the ester repeat unit to the ethylene oxide repeat unit of from 1 to 10, preferably from 2 to 6.

In a preferred embodiment the polyether-polyester copolymer is a multi-arm copolymer having from 3 to 8 arms.

If the polyether-polyester copolymer is a multi-arm copolymer the central core is a multi-arm polyether which may be obtainable from poly(ethylene glycol) (PEG) and a polyol. The multi-arm polyether may be formed by reaction of ethylene oxide with a polyol. The multi-arm polyether is obtainable by reaction of ethylene oxide with a polyol.

A polyol is an organic compound comprising a plurality of hydroxyl groups. Preferably, the polyol comprises at least three hydroxyl groups, optionally wherein the polyol is a hydrocarbon substituted with at least three hydroxyl groups, optionally 3, 4, 5, 6 or 8 hydroxyl groups. Typically the polyol is pentaerythritol (PE), dipentaerythritol, trimethylolpropane (TMP), glycerol, erythritol, xylitol, di(trimethylolpropane (diTMP) sorbitol, or inositol.

In one embodiment the polyol further comprises one or more ether groups.

Examples of multi-arm polyethers are presented in formula 1 to 4:

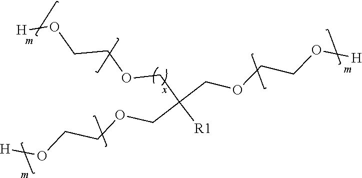

wherein R₁ is

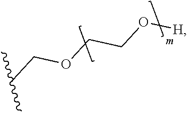

H or alkyl, x is 0 or 1 and m is an integer between 2 and 76

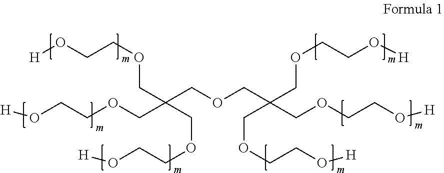

wherein m is an integer between 5 and 40

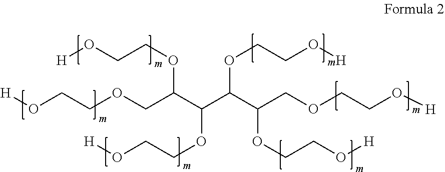

wherein m is an integer between 5 and 40

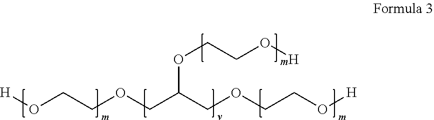

wherein m is an integer between 25 and 30 and v is 6

### Formula 4

In some embodiment for the multi-arm copolymer, the number of arms is 4, the molecular weight of the PEG core is 2 kDa, and the lactic acid/ethylene oxide molar ratio is 3 or 4.

Preferably the polyether-polyester copolymer is B(A)ₙ wherein B represents the polyether comprising PEG and A represents the polyester arms and n is an integer which is 1, 2, 3, 4, 5, 6, 7 or 8. When n is 1, the copolymer is a diblock, when n is 2, the copolymer is a triblock and when n is 3 or more, the copolymer is a multi-arm copolymer.

In the case of a diblock, the copolymer is linear and consists of a polyether and a polyester (A-B) such as mPEG-PLA, m representing an end-capping group such as methoxy.

In the case of a triblock the copolymer is linear and consists of a central polyether flanked by polyesters (A-B-A), such as PLA-PEG-PLA.

The molecular weight of the PEG chain, also referred to as the PEG repeat unit, namely —(CH₂CH₂O)ₙ— where n is an integer, is measured using gel permeation chromatography (GPC) using a calibration curve obtained from polystyrene standards. The molecular weight measured is the number average molecular weight (Mn).

General formulae for the diblock and triblock copolymers are set out below:

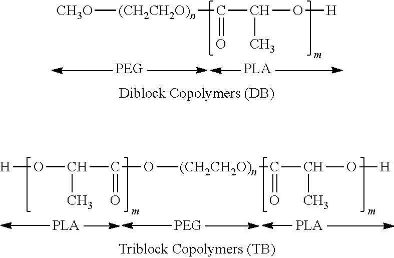

In preferred embodiments the total amount of the polyether-polyester copolymer is 2% (w/w) to 80% (w/w), optionally 10 to 50% (w/w), optionally 20 to 40% (w/w) of the total composition.

In one embodiment the polyether-polyester copolymer is a multi-arm copolymer and the amount of the multi-arm copolymer is from 20 to 60% (w/w), optionally 20 to 50% (w/w) of the total composition.

In a preferred embodiment the polyether-polyester copolymer is a mixture of a diblock copolymer and a triblock copolymer. In one embodiment the molar ratio of the ester repeat unit to the ethylene oxide repeat unit for the diblock copolymer is from 0.8 to 15, preferably from 1 to 10. In one embodiment the molar ratio of the ester repeat unit to the ethylene oxide repeat unit for the triblock copolymer is from 0.5 to 22, preferably from 0.5 to 10, most preferably from 1 to 6.

In some embodiments, for the triblock and/or the diblock copolymer the molecular weight of the PEG repeat unit is from 1 to 2 kDa and the lactic acid/ethylene molar ratio is from 2 to 6. In some embodiments, for the triblock copolymer the molecular weight of the PEG repeat unit is from 1 to 2 kDa and the lactic acid/ethylene ratio is 2 to 6 and for the diblock copolymer the molecular weight of the mPEG is from 1 to 2 kDa and the lactic acid/ethylene oxide ratio is from 3 to 4.

In some embodiments, for the triblock copolymer the molecular weight of the PEG repeat unit is 1 kDa, and the lactic acid/ethylene oxide molar ratio is 4 or 6 and for the diblock copolymer the molecular weight of the PEG repeat unit is 2 kDa, and the lactic acid/ethylene oxide molar ratio is 3.

In some embodiments, for the triblock copolymer the molecular weight of the PEG is 1 kDa, and the lactic acid/ethylene oxide molar ratio is 6 and for the diblock copolymer the molecular weight of the mPEG is 1 kDa, and the lactic acid/ethylene oxide molar ratio is 4.

In some embodiments, for the triblock copolymer the molecular weight of the PEG is 2 kDa, and the lactic acid/ethylene oxide molar ratio is 2 and for the diblock copolymer the molecular weight of the mPEG is 2 kDa, and the lactic acid/ethylene oxide molar ratio is 3.

Typically the amount of the diblock copolymer is from 2 to 30% (w/w), optionally 10 to 30% (w/w), optionally 10 to 20% (w/w) of the total composition; and the amount of the triblock copolymer is from 2 to 30% (w/w), optionally 10 to 30% (w/w), optionally 10 to 20% (w/w) of the total composition.

Preferably:
when the copolymer is a multi-arm copolymer B(A)ₙ, each polyether arm is composed of 2 to 150 ethylene oxide repeat units and each polyester arm A is composed of 4 to 200 ester repeat units, with a preferred molar ratio of the ester repeat unit to the ethylene oxide repeat unit in the multi-arm copolymer ranging from 1 to 10 and more preferably 2 to 6;
when the copolymer is a triblock copolymer A-B-A, B is composed of 3 to 300 ethylene oxide repeat units and each A arm is composed of 1 to 3,000 ester repeat units, with a preferred molar ratio of the ester repeat unit to the ethylene oxide repeat unit in the triblock copolymer ranging from 0.5 to 22, preferably 0.5 to 10 and more preferably 1 to 6; and
when the copolymer is a diblock copolymer A-B, B is composed of 2 to 250 ethylene oxide repeat units and A is composed of 1 to 3,000 ester repeat units, with a preferred molar ratio of the ester repeat unit to the ethylene oxide repeat unit in the diblock copolymer ranging from 0.8 to 15 and more preferably 1 to 10.

More details on the copolymers used in the present invention can be found in WO2012/090070A1, WO2019016233A1, WO2019016234A1, WO2019016236A1 and WO2020/144239A1 incorporated by reference herein.

The triblock PLA-PEG-PLA polymers described herein are labelled PxRy, where x represent the size of the PEG chain in kDa (number average molecular weight) and y is the LA/EO molar ratio. The diblock mPEG-PLA polymers described herein are labelled dPxRy where x represents the size of the PEG chain in kDa (number average molecular weight) and y is the LA/EO molar ratio. The star-shaped sPEG-PLA polymers described herein are labelled szPxRy where x represents the size of the PEG chain in kDa (number average molecular weight), y is the LA/EO molar ratio and z the arm number.

The acidic compound has a pKₐ in water (pKₐ(H₂O)) of less than 3.00. Each acidic compound preferably has a a pKₐ(H₂O) of from −15.00 to 2.97, more preferably from about −3.00 to about 2.90, optionally from about 0.50 to about 2.75, optionally from about 1.40 to about 2.75.

pKₐ is the negative log of the acid dissociation constant or Kₐ value. The pKa is determined at a fixed temperature, typically 25° C. The pKₐ of a compound is a measure of the strength of an acid in a given solution, i.e., its capacity to release a free proton in solution and is thus specific to the solution. It can be defined upon following chemical reaction:

HA<==>A⁻+H⁺

with HA being the acid, A⁻ the deprotonated acid and H⁺ a free proton.

It is calculated according to following formula:

$$p {K}_{a} = - log ( \frac{[ {A}^{-} ] [ {H}^{+} ]}{[ AH ]} )$$

with [X] being the concentration of compound X in solution at equilibrium.

Thus, the lower the pKₐ, the higher the concentration of free protons in solutions.

Examples of acids with pKₐ(H₂O) lower than 3 are aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid, tartaric acid citraconic acid, methylphosphonic acid, ethylphosphonic acid, propylphosphonic acid, butylphosphonic acid, pentylphosphonic acid, hexylphosphonic acid, heptylphosphonic, octylphosphonic acid, nicotinic acid, hydroiodic acid, chromic acid, trifluoromethane sulfonic acid, trichloroacetic acid, dichloroacetic acid, bromoacetic acid, chloroacetic acid, cyanoacetic acid, 2-chloropropanoic acid, 2-chlorobutanoic acid, 4-cyanobutanoic acid, perchloric acid, a phosphoric acid or a combination thereof.

Preferred acids are aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid or tartaric acid.

In another embodiment, the acidic compound is an acid with a pKₐ in dimethyl sulfoxide (DMSO), (pKₐ(DMSO)), lower than 10, preferably lower than 8. Recent computational chemistry studies allow the pKₐ of acids in various solvents to be calculated (Empirical conversion of pKₐ values between different solvents and interpretation of the parameters: application to water, acetonitrile, dimethyl sulfoxide, and methanol, E. Rossini, D. Bocherarov and E. W. Knapp. ACS Omega; 2018; Computing pKₐ values in different solvents by electrostatic transformation, E. Rossini and E. W. Knapp. Journal of Chemical Theory and Computation; 2016).

Examples of acids with pKₐ(DMSO) lower than 10 are gentisic acid, hydrochloric acid, oxalic acid, sulfamic acid or sulfonic acid.

In one embodiment the protective acidic compound is a carboxylic acid, optionally a polycarboxylic acid, optionally a di or tricarboxylic acid.

In another embodiment, the protective acidic compound is an inorganic acid.

In one embodiment, the protective acidic compound is selected from the list consisting of salicylic acid, oxalic acid, malonic acid, sulfamic acid, pamoic acid or combination thereof.

The acidic compound should be present in an amount which is sufficient to prevent nucleophilic induced polyester degradation, but which is low enough to avoid promoting acid catalyzed polymer degradation. The acidic compound is referred to as a protective acidic compound because it protects the copolymer from degradation. Numerous studies have demonstrated the impact of pH on copolymer degradation, a low pH promoting the protonation of the polyester and increasing the occurrence of nucleophilic attack (Hydrolytic degradation and erosion of polyester biomaterials, L. N. Woodard and M. A. Grunlan. ACS Macro Letters; 2018. Biodegradation of aliphatic polyesters, S. Li and M. Vert, in Degradable Polymers: Principles and Application, Kluwer academic publishers; 2002).

In some embodiments the amount of the at least one acidic compound is from 0.005% (w/w) to 10% (w/w), optionally 0.55% (w/w) to 10% (w/w), or 0.005% (w/w) to 0.45% (w/w), preferably 0.01% (w/w) to 4.0% (w/w) of the total composition.

As used herein the term nucleophilic compound refers to a molecule comprising at least one nucleophilic group capable of cleaving ester bonds of the polyester which results in polymer fragmentation and thus in polymer and formulation degradation. Nucleophilic groups capable of attacking the polymer are groups presenting a pair of electrons that can react with an electrophile or an electrophilic center. An electrophilic center is commonly defined as the element of a polar compound that is the most electron deficient. Typical nucleophilic groups include groups with a mobile hydrogen atom.

The person skilled in the art would know how to identify a nucleophilic compound and therefore, the present invention is not limited to the cited examples nor to any particular nucleophile.

Typically the nucleophilic compound comprises one or more functional groups selected from —SH, —OH, a primary amine (—NH₂), a secondary amine (—NRH), a tertiary amine (—NRR′), a heterocyclic group, and combinations thereof.

In one embodiment the nucleophilic compound is an active pharmaceutical ingredient. In an alternative embodiment the composition comprises an active pharmaceutical ingredient and a separate nucleophilic compound. The nucleophilic compound may be a solvent, a co-solvent, a solubility enhancer, a porogen, or a phase exchange modifier.

When the compositions of the invention contain an API, they provide sustained release of the API. The term “sustained release” means that the active pharmaceutical ingredient can be released gradually over an extended period of time. This sustained release may be linear or non-linear and typically can last between several days to 1 year or more depending on the pharmaceutical composition and the amount of it administered.

“Pharmaceutically active ingredient” means a drug or medicine for treating, preventing and/or ameliorating a medical condition, illness or disease or symptoms thereof. For the purposes of the present application the term “active principle” has the same meaning as “active ingredient”. Thus, the terms active ingredient, active principle, drug, or medicine are used interchangeably. The term Active Pharmaceutical Ingredient, or “API” is also used. The term drug or active ingredient as used herein includes without limitation physiologically or pharmacologically active substances that act locally or systemically in the body of an animal or plant.

The pharmaceutically effective amount of a pharmaceutically active ingredient may vary depending on the pharmaceutically active ingredient, the extent medical condition of the animal or plants and the time required to deliver the pharmaceutically active ingredient. There is no critical upper limit on the amount of pharmaceutically active ingredient incorporated into the polymer solution as long as the solution or suspension has a viscosity which is acceptable for injection through a syringe coupled with a needle and that it can effectively treat the medical condition without subjecting the animal or plant to an overdose. The lower limit of the pharmaceutically active ingredient incorporated into the delivery system is dependent simply upon the activity of the pharmaceutically active ingredient and the length of time needed for treatment.

In one embodiment the active pharmaceutical ingredient is a free base or is a salt of an acid having a pKₐ(H₂O) of greater than 3. In one embodiment, the active pharmaceutical ingredient is octreotide acetate, liothyronine, escitalopram free base, atorvastatin calcium trihydrate or a combination thereof.

The active pharmaceutical ingredient may also be other active principle comprising at least one nucleophilic group such as SH, —OH, a primary amine (—NH₂), —NRH (a secondary amine), —NRR′ (a tertiary amine), a heterocyclic group, wherein each R and each R′ are independently a C₁ to C₁₀ hydrocarbyl group, or combinations thereof.

The active pharmaceutical ingredient may be a peptide, polypeptide or a protein.

In one embodiment the nucleophilic compound is an alcohol, optionally a C₁ to C₈ alcohol, optionally glycerol, sorbitol, methanol, ethanol, propanediol, propylene glycol, polyethylene glycol, preferably methanol, propylene glycol, polyethylene glycol or mixtures thereof.

In one embodiment the nucleophilic compound is a saccharide, disaccharide or polysaccharide, optionally sucrose, dextrose, cyclodextrin, chitosan or mixtures thereof.

In one embodiment the nucleophilic compound is an amino acid, peptide, polypeptide or protein, optionally lysine, arginine, histidine or serine.

In one embodiment the nucleophilic compound is water.

In one embodiment the nucleophilic compound is a further organic solvent, i.e. a solvent in addition to the at least one organic solvent defined in c) above, optionally pyrrolidone-2, glycofurol, pyridine, nitromethane, triethylamine, N,N-dimethylaniline, N,N-diemthyldecanamide, N,N-dimethyloctanamide, 2,4,6-collidine or mixtures thereof.

In one embodiment the composition comprises at least one active pharmaceutical ingredient and the nucleophilic compound is a solubility enhancer, a porogen or a phase exchange modifier.

A solubility enhancer improves the solubility of the active pharmaceutical ingredient within the composition.

The solubility enhancer can be a cosolvent together with the biodegradable organic solvent c) or a solid compound which is soluble in it.

A solubility enhancer can be a further organic solvent or cosolvent selected from the group consisting of benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, triacetin, tributyrin, tripropionin, glycofurol, pyridine, nitromethane, trimethylamine, N,N-dimethylaniline, N,N-dimethyldecanamide, N,N-dimethyloctanamide, 2,4,6-collidine and mixtures thereof.

In one embodiment the solubility enhancer is selected from the list consisting of propylene glycol, polyethylene glycol, glycerol, sorbitol, a cyclodextrin and mixtures thereof.

In another embodiment, the nucleophilic compound acts as a porogen, modifying the formation of pores within the in situ forming depot.

A porogen can act on the active pharmaceutical ingredient and/or solvent release from the in situ forming depot by impacting the size and/or the number of pores within the depot. Typically, porogens are compounds in suspension that will dissolve upon depot formation leaving pores within the depots that will promote diffusion out of the depot, typically diffusion of the API.

The release profile of the active pharmaceutical ingredient may be modulated through the incorporation of such a compound within the composition In another embodiment, the nucleophilic compound is a phase exchange modifier, modulating the exchange of the organic solvent between the in situ forming depot and the surrounding media. A phase exchange modifier can impact the active pharmaceutical release from the in situ formed depot by modifying the solvent exchange with surrounding media and thus the resulting microstructure of the depot.

Examples of porogens or phase exchange modifiers are saccharides, disaccharides or polysaccharides, such as sucrose or dextrose, or fatty acids, such as triglyceride, or vegetable oils, or alcohols, such as a C₁ to C₈ alcohols or polyethylene glycol.

In one embodiment the porogen or the phase exchange modifier is selected from the list consisting of saccharides, polysaccharides or alcohols.

The nucleophilic compound may be selected from the list consisting of octreotide acetate, liothyronine, escitalopram free base, atorvastatin calcium trihydrate, PEG1000, methanol, propylene glycol or a mixture thereof.

The compositions of the invention comprise at least one organic solvent. The organic solvent is a pharmaceutically acceptable solvent or a biocompatible solvent. The solvent is suitable for administration to human or non-human animals. Typically the at least one organic solvent c) is selected from the group consisting of benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, triacetin, tributyrin, tripropionin, glycofurol or a mixture thereof, preferably DMSO, NMP and mixtures thereof.

The molar amount of the acidic compound may be 0.05% to 300% relative to the molar amount of the nucleophilic compound, preferably 0.1% to 250%. In one embodiment the nucleophilic compound contains at least one —OH group and the molar amount of the acidic compound is equal to or lower than 100% relative to the molar amount of the nucleophilic compound, preferably 0.05% to 100% relative to the molar amount of the nucleophilic compound. In one embodiment the nucleophilic compound contains at least one nitrogen containing reactive group such as a primary amine or a secondary amine, and the molar amount of the acidic compound is equal to or greater than 100% relative to the molar amount of the nucleophilic compound, preferably 100% to 300% relative to the molar amount of the nucleophilic compound. The relative amounts of the acidic and nucleophilic compounds can also be expressed as a molar ratio as set out in the examples.

Typically the amount of the active pharmaceutical ingredient is 0.05% (w/w) to 60% (w/w), optionally 0.05 to 20% (w/w), optionally 0.05 to 10% (w/w), optionally 0.05 to 5% (w/w), optionally 0.05 to 2% (w/w) of the total composition.

Typically the amount of the organic solvent is at least 20% (w/w) of the total composition, optionally 20 to 80% (w/w), optionally 20 to 60% (w/w).

The compositions of the invention are suitable for parenteral administration. The term “parenteral administration” encompasses intramuscular, intraperitoneal, intra-abdominal, subcutaneous, intravenous and intraarterial. It also encompasses intradermal, intracavernous, intravitreal, intracerebral, intrathecal, epidural, intra-articular, and intraosseous administration. The pharmaceutical composition is preferably suitable for parenteral administration.

In a preferred embodiment the compositions are injected using a needle and syringe, optionally using an injection device. Typical volumes of injection of the composition administered to a subject are 0.05 mL to 5 mL or 0.1 to 1.5 mL.

The subject may be an animal or a plant. The term “animals” encompasses all members of the Kingdom Animalia. The animal may be a human or non-human animal.

As used herein the term “plant” encompasses all members of the Plant Kingdom.

In preferred embodiments the composition is stable for at least 2 weeks of storage at room temperature or 2 to 8° C., preferably at least 4 weeks of storage at room temperature or 2 to 8° C.

The stability of the composition can be measured by determining the dynamic viscosity of the composition over time, since degradation of the copolymer leads to smaller copolymer fragments that can impact the overall composition viscosity.

The stability of the composition can be measured by determining the concentration of the API over time, since interactions between the API and copolymers or copolymers degradation by-products can induce a loss in native API.

The stability of the composition over time can also be measured by visual observation, for example by observing the colour of a composition relative to a standard.

Alternatively, the stability of the composition can also be measured by performing GPC analysis of the composition over time, since degradation of the copolymer leads to smaller copolymer fragments, impacting copolymer molecular weight distribution.

In one embodiment the concentration of the active pharmaceutical ingredient in the composition reduces by less than 20%, preferably less than 10%, more preferably less than 5% after 2 weeks of storage at room temperature or 2 to 8° C., preferably 4 weeks of storage at room temperature or 2 to 8° C. relative to the initially formulated composition.

In one embodiment the dynamic viscosity of the composition reduces by less than 10%, preferably less than 5% after 2 weeks of storage at room temperature or 2 to 8° C., preferably 4 weeks of storage at room temperature or 2 to 8° C. relative to the initially formulated composition.

In a further aspect of the invention, provided is method for preparing a pharmaceutical composition as described above comprising or consisting of the steps of:
i. dissolving the at least one polyether-polyester copolymer a) as defined above in the at least one organic solvent c);
ii. adding to the product of step i) at least one acidic compound d) as defined above and at least one nucleophilic compound b) as defined above, optionally wherein the nucleophilic compound b) is an active pharmaceutical ingredient; and
iii. homogenizing the product of step ii), thereby obtaining the pharmaceutical composition.

In a preferred embodiment the at least one acidic compound and the at least one nucleophilic compound do not form a salt or complex prior to step ii). In an embodiment of the invention, the at least one acidic compound and the at least one nucleophilic compound are not contacted or mixed together prior to step ii). A great advantage of the present invention over prior art methods is that no initial step is required in which the acidic compound is reacted with the nucleophilic compound (which may be an API) before the nucleophilic compound is mixed with the other components of the composition, in particular the copolymer. In an embodiment of the present invention, all of the reactants can be mixed together in a single step, and the acid can achieve its stabilization effect without first having to be reacted with the nucleophilic compound.

In a preferred embodiment step ii) consists of mixing the components in a single step.

In another aspect, the invention provides a method for preparing a pharmaceutical composition as described above comprising or consisting of the steps of:
i. dissolving the at least one polyether-polyester copolymer a) as defined in any preceding claim in the at least one organic solvent c);
ii. adding to the product of step i) at least one acidic compound d) as defined above or at least one nucleophilic compound b) as defined above, and then homogenizing the product;
iii. if at least one acidic compound d) is added in step ii) then subsequently adding at least one nucleophilic compound b) as defined above; or if at least one nucleophilic compound b) is added in step ii) then subsequently adding at least one acidic compound d) as defined above; and
iv. homogenizing the product of step iii), thereby obtaining the pharmaceutical composition; optionally wherein the nucleophilic compound b) is an active pharmaceutical ingredient.

In other words, in step ii) the at least one acidic compound d) is added to the product of step i) and after homogenization the nucleophilic compound b) is added to the composition or the reverse steps occur so that compound b) is added then compound d). This also limits or avoids the initial formation of a salt or complex of the nucleophilic compound.

In embodiments of the invention, the nucleophilic compound is not an active pharmaceutical ingredient and an active pharmaceutical ingredient is added after step i).

In one embodiment the active pharmaceutical ingredient is previously dissolved in the organic solvent. In one embodiment the acidic compound is previously dissolved in the organic solvent. In one embodiment the nucleophilic compound is previously dissolved in the organic solvent. These embodiments are beneficial because it may be that the target concentration of the active pharmaceutical ingredient, acidic compound or nucleophilic compound is too low to allow accurate weighing of the agent. Therefore a concentrate of higher concentration is prepared. Alternatively or in addition the viscosity of the vehicle of step i) might have led to difficulty of homogenizing the composition. By first dissolving an amount of the active pharmaceutical ingredient, acidic compound or nucleophilic compound in the solvent, we thereby obtain a first homogeneous solution or suspension that can then be more easily mixed with the vehicle of step i).

In one embodiment the pharmaceutical composition obtained in step iii. or iv. is filtered.

In a further aspect, provided is a pharmaceutical composition obtainable or obtained by the method defined above.

The homogenization of the formulation may be obtained by placing the container on a roller mixer or on a magnetic stirrer.

The polymeric vehicle or the pharmaceutical composition may be filtered, preferably sterilized by filtration. Alternative methods of sterilization may be used by a skilled person in the field.

In a further aspect, provided is a pharmaceutical composition obtainable or obtained by the method defined above.

“Viscosity,” by definition and as used herein, is a measure of a fluid's resistance to flow and gradual deformation by shear stress or tensile strength. It describes the internal friction of a moving fluid. For liquids, it corresponds to the informal concept of “thickness”. By “dynamic viscosity” is meant a measure of the resistance to flow of a fluid under an applied force. The person skilled in the art would understand that the degradation of the polyester part within the pharmaceutical composition of the invention would induce a change of its dynamic viscosity. In particular, the generation of smaller polyester chains would typically induce a decrease of the dynamic viscosity of the pharmaceutical composition.

Dynamic viscosity is determined using an Anton Paar Rheometer equipped with cone plate measuring system. Typically, around 700 μL of studied formulation are placed on the measuring plate. The temperature is controlled at +25° C. The measuring system used is a cone plate with a diameter of 50 mm and a cone angle of 1 degree (CP50-1). The working range is from 10 to 1000 s⁻¹. Formulations are placed at the center of the thermo-regulated measuring plate using a positive displacement pipette. The measuring system is lowered down and a 0.104 mm gap is left between the measuring system and the measuring plate. 21 viscosity measurement points are determined across the 10 to 1000 s⁻¹ shear rate range. Given values are the ones obtained at the middle of the plateau of the curve, which is representative of the viscosity profile, typically 100 s⁻¹.

The dynamic viscosity of the initially formulated composition measured at 25° C. is typically 1 to 5000 mPa·s, preferably 1 to 2000 mPa·s, more preferably 10 to 500 mPa·s or 500 to 2000 mPa·s.

The active pharmaceutical ingredient amount or concentration, also referred to as “drug content”, or “assay”, is the concentration of active pharmaceutical ingredient within the pharmaceutical composition and is represented in weight percentage (% w/w) of the total composition. It can be calculated as a percentage recovery of theoretical active pharmaceutical ingredient, based on masses recorded during composition preparation. It can also be normalized to the content measured after formulation reconstitution.

The amount or concentration of the active pharmaceutical ingredient can be measured using a liquid chromatography system. The elution conditions and columns used, must be adapted to the active pharmaceutical ingredient but would be well-known to a skilled person. Typically, a Waters Acquity UPLC system with a UV detector and analytical column obtained from Waters can be used.

A stable pharmaceutical composition should present drug content and dynamic viscosity values with less than 10% variation compared to the initial analyses, preferably, less than 5% variation.

In one embodiment, the pharmaceutical composition of the invention is stable for at least 2 weeks after its preparation under storage conditions, preferably at least 4 weeks.

Typically, compositions of the invention are stored at room temperature (20 to 25° C.) or under refrigerated conditions (2 to 8° C., typically 4° C.) after preparation.

Wherever embodiments are described with the language “comprising,” otherwise analogous embodiments described in terms of “consisting of” and/or “consisting essentially of” are included.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### BRIEF DESCRIPTION OF THE DRAWINGS

FIG. 1: Evolution of octreotide content of formulations F19, F20, F21, F22, F23 and F24 after 2 days at room temperature. Drug recovery was determined as described in example 3. Results show that the copolymer content only affects the results variability but not the drug recovery over time. Pamoic acid significantly reduces the API degradation. No differences are observed between the 2 acid contents tested over this period.

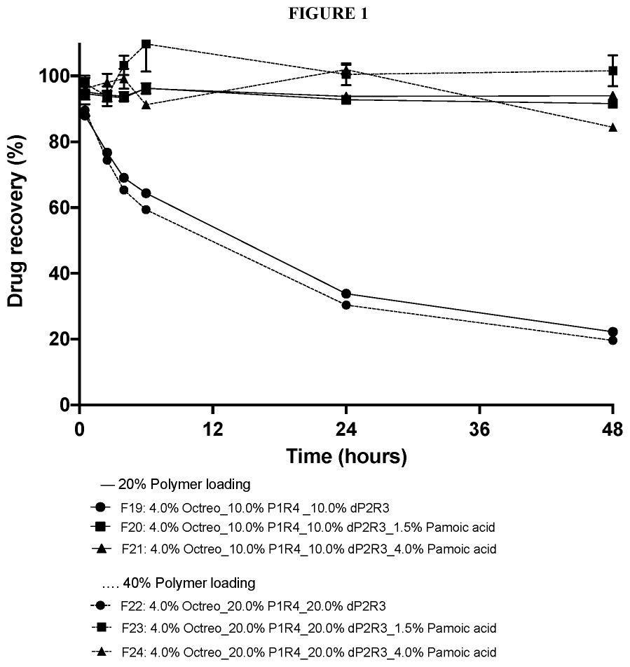

FIG. 2: Evolution of octreotide content of formulations F22, F30, F31, F34 and F35 after 10 days at room temperature. Drug recovery was determined as described in example 3. Results show that the addition of sodium dodecyl sulfate (SDS), docusate, sucrose acetate isobutyrate (SAIB) or butylene hydroxytoluene (BHT) has no effect on drug recovery over time.

FIG. 3: Evolution of octreotide content of formulations F22, F23, F32, F33, F37, F38 and F53 after 10 days at room temperature. Drug recovery was determined as described in example 3. Data indicate that the addition of an acid into the formulation increases the drug recovery compared to the control formulation. At a fixed equimolar octreotide/acid ratio, different levels of recoveries are obtained depending on the chosen acid, pamoic and oxalic acids presenting the highest drug recoveries with time.

FIG. 4: Evolution of octreotide content of formulations F22, F23, F33, F37, F49, F50 and F51 after 10 days at room temperature. Drug recovery was determined as described in example 3. Data demonstrate that with the 3 tested acids (pamoic, formic and oxalic), the acid content has an impact on octreotide recovery. A higher peptide recovery is measured with a higher acid loading.

FIG. 5: Evolution of octreotide content of formulations F22, F23 and F52 after 10 days at room temperature. Drug recovery was determined as described in example 3. Results demonstrate that the protonation state of the acid highly impacts the octreotide recovery over time, with the pamoate salt inducing the same drug recovery level as the control formulation.

FIG. 6: Evolution of the viscosity of formulations F17, F18, F25, F26, F27 and F39 after 1 month and 2 months at 40° C. A forced degradation study was performed as described in example 3. Results show that the viscosity of control octreotide formulation (F39) has dropped from half its initial level after 1 month at 40° C., and even more in presence of propylene glycol (F27), whereas in presence of pamoic acid, the viscosity decrease is reduced.

FIG. 7: Evolution of octreotide content of formulations F17, F18, F25, F26, F27 and F39 after 1 month and 2 months at 40° C. Drug recovery was determined as described in example 3. Data indicate that while no native peptide could be detected after 1 month at 40° C. for the control formulation (F39) or formulation containing propylene glycol alone (F27), over 80 and 90% are recovered after 2 months in the presence of 1.5 or 5% of pamoic acid, with or without propylene glycol.

FIG. 8: Evolution of the octreotide content of formulations F122, F123 and F124 after 2 and 4 weeks at 4° C. Drug recovery was determined as described in example 3. Results show that for formulations with oxalic acid, drug recovery is stable and close to 100% during the full length of the study, while for control formulation F123, drug content is below 30% after only 2 weeks.

FIG. 9: In vitro release profiles of formulation F123 at study start and after 2 and 4 weeks at 4° C. Stability study and IVR tests were performed as described in example 3. API release was normalized according to the drug content measured at the corresponding timepoint. Different profiles are obtained at each timepoint. After 2 and 4 weeks of storage, the remaining API is released faster from the depots due to formulation degradation.

FIG. 10: Evolution of Liothyronine content of formulations F32, F46, F47 and F48 over 24 hours at RT. Drug content was measured as disclosed in example 4. Results show that for control formulation F32 or formulation F48 with CaCl₂), drug recovery starts decreasing 3 hours after formulation reconstitution. In the presence of acid (oxalic or pamoic, F46 and F47 respectively), drug recovery is stable up to at least 24 hours.

FIG. 11: Evolution of Liothyronine content of formulations F39, F50, F51, F52, F53 F54, F55 and F56 after 7 days at RT. Drug content was measured as disclosed in example 4. Data show that oxalic acid contents from 0.025 to 0.50% have an impact on drug recovery level compared to control formulation F39. In particular with 0.25 and 0.50% of oxalic acid (F55 and F56), API contents remain close to 95% of their initial values up to at least 7 days.

FIG. 12: Evolution of Liothyronine content of formulations F57, F58 and F59 after up to 2 weeks of storage at RT or 4° C. A stability study was performed as disclosed in example 4. Results indicate that while a decrease in drug recovery is observed with control formulation F57 stored at RT or 4° C., API contents of formulation F58 and F59 remain close to 95% of their initial values in the presence of 0.10 or 0.25% of oxalic acid.

FIG. 13: Evolution of the viscosity of vehicles V55, V56, V58, V59, V61, V62, V64 and V65 after 2 and 4 weeks at 50° C. A forced degradation study was performed as disclosed in example 5. Results show that whatever the structure of the copolymers (linear or star, V58 and V59 respectively), a decrease of viscosity is observed with time in presence of propylene glycol. However, when further adding pamoic acid into the vehicles (V64 and V65), the viscosity decrease is reduced. A light viscosity decrease is observed in the presence of the acid alone (V61 and V62).

FIG. 14: Evolution of the viscosity of vehicles V54, V57, V60 and V63 after 2 and 4 weeks at 50° C. A forced degradation study was performed as disclosed in example 5. As for FIG. 13, at a lower copolymer content, the viscosity decrease induced by propylene glycol (V57) is significantly reduced by the addition of pamoic acid (V63). A light viscosity reduction is noticed in presence of pamoic acid only (V60).

FIG. 15: Evolution of the viscosity of vehicles V55, V58, V61, V64, V66, V67, V71, V72, V86, V87, V92 and V93 after 2 and 4 weeks at 50° C. A forced degradation study was performed as disclosed in example 5. Results demonstrate that in presence of pamoic acid alone at a concentration equal to or lower than 0.26% (w/w %), vehicles are stable. Moreover, in the simultaneous presence of propylene glycol and pamoic acid, the propylene glycol induced polymer degradation is significantly reduced, even at low pamoic acid contents.

FIG. 16: Evolution of the viscosity of vehicles V55, V58, V68, V69, V70, V73, V74, V75, V88, V89, V94 and V95 after 2 and 4 weeks at 50° C. A forced degradation study was performed as disclosed in example 5. Results demonstrate that in presence of oxalic acid alone at a concentration equal to or lower than 0.1% (w/w %), vehicles are stable. Moreover, in the simultaneous presence of propylene glycol and oxalic acid, the propylene glycol induced polymer degradation is significantly reduced, even at low oxalic acid contents, such as 0.01% (w/w %) (V95) for which the composition is stable.

FIG. 17: Evolution of the viscosity of vehicles V55, V58, V78, V82, V90, V91, V96 and V97, after 2 and 4 weeks at 50° C. A forced degradation study was performed as disclosed in example 5. Results demonstrate that in presence of salicylic acid alone at a concentration equal to or lower than 0.18 w/w %, vehicles are stable. Moreover, in the simultaneous presence of propylene glycol and salicylic acid, the propylene glycol induced degradation is highly reduced.

FIG. 18: Evolution of the viscosity of vehicles V68, V73, V76, V77, V78, V79, V80, V81, V82, V83, V84 and V85, after 2 and 4 weeks at 50° C. A forced degradation study was performed as disclosed in example 5. Data indicate that at close weight concentration, acids of different pKₐ have similar impact on vehicles viscosity. However, when further adding propylene glycol at a fixed acid/propylene glycol molar ratio, the acids have different impact on vehicles viscosity. The lower the pKₐ (as disclosed in table 1), the lower the viscosity decrease in presence of propylene glycol.

FIG. 19: Evolution of the viscosity of vehicles V66, V68, V71, V73, V77, V78, V80, V82, V83 and V85, after 2 and 4 weeks at 50° C. A forced degradation study was performed as disclosed in example 5. Data indicate that at fixed acid/propylene glycol molar ratio, similar results are obtained with pamoic, salicylic, sulfamic and oxalic acids. A higher viscosity decrease is observed in the presence of propylene glycol and malonic acid, the latter having the highest known pKₐ(DMSO) from those acids.

FIG. 20: Evolution of the viscosity of vehicles V103, V105, V106 and V107, loaded with oxalic acid contents equivalent to acid/PEG1000 molar ratios of 0; 0.1/100; 1/100 or 5/100 respectively; after 2 and 4 weeks at 50° C. A forced degradation study was performed as disclosed in example 5. Data indicate that the polymer degradation induced by PEG1000 is reduced in presence of oxalic acid and that vehicle with 0.01% (w/w %) of oxalic acid do not present any degradation evidence at the end of the study.

FIG. 21: Evolution of the viscosity of vehicles V104, V108, V109 and V110, loaded with oxalic acid contents equivalent to acid/MeOH molar ratios of 0; 0.1/100; 1/100 or 5/100 respectively; after 2 and 4 weeks at 50° C. A forced degradation study was performed as disclosed in example 5. Data indicate that the polymer degradation induced by MeOH is highly reduced in presence of oxalic acid, notably in formulations containing 0.03% (w/w %) of acid. With higher amounts of oxalic acid, higher viscosity decreases are measured.

FIG. 22: Evolution of viscosity of formulations F111, F112, F114, F115, F116, F117, F118 and F119 after 1 and 2 weeks at 80° C. A forced degradation study was performed as disclosed in example 6 and viscosity were normalized to values at study start. Data indicates that all formulations whatever the escitalopram form or oxalic acid content present a strong decrease of viscosity. Compared to control escitalopram free base formulation F111, the addition of acid in a molar ratio between 0.5/1 and 2/1 reduces the viscosity decrease. In particular, formulation F118 with oxalic acid in molar ratio of 1.5/1 presents the lowest viscosity decrease.

FIG. 23: Evolution of viscosity of formulations F111, F112 and F118 after 2 and 4 weeks at RT or 4 weeks at 4° C. A stability study was performed as disclosed in example 6. Results show that the addition of oxalic acid is reducing the escitalopram induced degradation and that viscosity decreases of formulations F112 (with escitalopram oxalate) and F118 are similar. Moreover, decreasing storage temperature, slow down the viscosity decrease.

FIG. 24: In vitro release profiles of formulation F111 at study start and after 2 or 4 weeks of storage at RT. IVR tests were conducted as disclosed in example 6. API release was normalized according to the drug content measured at the corresponding timepoint. Data show that a slight acceleration is observed from 2 days after 2 and 4 weeks of storage of F111.

FIG. 25: Evolution of atorvastatin contents of formulations F125, F126, F127, F128, F129 and F130 after 1 and 2 weeks of storage at 50° C. A forced degradation study was performed as detailed in example 7. Results indicate that the addition of 0.70 to 1.40% of oxalic acid increased the atorvastatin recovery with time. On the contrary, lower or higher contents decreased the atorvastatin recovery.

FIG. 26: Evolution of atorvastatin contents of formulations F125, F128, F129, F132, F133, F134 and F135 after 1 and 2 weeks of storage at 50° C. A forced degradation study was performed as detailed in example 7. Results indicate that by increasing the oxalic/atorvastatin molar ratio up to a 90/100 (1.26% oxalic acid), an increase of API recovery is observed. Above this threshold, a lower API content is measured with time.

FIG. 27: Evolution of atorvastatin contents of formulations F125, F131, F134, F136, F137, F138, F143 and F144 after 1 and 2 weeks of storage at 50° C. A forced degradation study was performed as detailed in example 7. Results indicate that the simultaneous presence of atorvastatin and PEG-PLA copolymers is inducing the API degradation, with recoveries lower than 30% of the initial API content. In the presence of a fixed amount of oxalic acid, whatever the polymer type and/or structure, an increase of drug recovery is observed with recoveries close to 60%.

FIG. 28: Evolution of atorvastatin contents of formulations F125, F134, F139 and F140 after 1 and 2 weeks of storage at 50° C. A forced degradation study was performed as detailed in example 7. Results indicate that different levels of degradation are obtained depending on solvent type, but that oxalic acid reduces atorvastatin degradation in both DMSO and NMP.

FIG. 29: Evolution of atorvastatin contents of formulations F125, F135, F141 and F142 after 1 and 2 weeks of storage at 50° C. A forced degradation study was performed as detailed in example 7. Results indicate that the API degradation kinetics is impacted by the initial API loading and that the oxalic acid/API ratio needs to be adjusted depending on the initial API content.

FIG. 30: Evolution of atorvastatin contents of formulations F125, F134 and F135 after 2 and 4 weeks of storage at room temperature. A stability study was performed as detailed in example 7. Results indicate that the addition of oxalic acid within the formulation leads to drug recovery of over 95% while less than 65% of the API were recovered in the control formulation.

FIG. 31: Evolution of the viscosity of formulations F125, F134 and F135 after 2 and 4 weeks of storage at room temperature. A stability study was performed as detailed in example 7. Results indicate that the addition of oxalic acid induces less viscosity decrease overtime. Interestingly, a clear difference can be observed between F134 and F135, whose compositions only differ in 0.14% oxalic acid.

FIG. 32: Evolution of the release profile of formulation F125 after 2 and 4 weeks of storage at room temperature. A stability study was performed as detailed in example 7. API release was normalized according to the drug content measured at the corresponding timepoint. Different profiles are obtained at each timepoint. After 4 weeks of storage, the remaining API is released faster, and a higher variability is observed due to a fragility of the depots made from the degraded formulation.

FIG. 33: Rat plasma concentration profiles after subcutaneous administration of octreotide formulations F162 and F165. An in vivo PK study was performed as described in example 8. Results indicate that similar octreotide sustained release profiles were obtained in rats over 336 hours with the 2 tested formulations.

FIG. 34: Rat plasma concentration profiles after subcutaneous administration of octreotide formulations F122 and F123. An in vivo PK study was performed as described in example 9. Results indicate that sustained releases of octreotide were obtained in rats over 240 hours with the 2 tested formulations.

The invention will now be described further with reference to the following clauses:

1. A pharmaceutical composition comprising or consisting of
a) at least one polyether-polyester copolymer, wherein the copolymer has the formula:

B(A)ₙ
wherein B represents a polyether and comprises polyethylene glycol (PEG), each A represents a polyester arm and n is an integer from 1 to 8;
b) at least one nucleophilic compound;
c) at least one organic solvent; and
d) up to 10% (w/w) of at least one acidic compound having a pKₐ(H₂O) of less than 3.

2. A pharmaceutical composition according to clause 1 wherein the at least one polyether-polyester copolymer a) is selected from;
i. a multi-arm copolymer having 3 to 8 polyester arms attached to a central core which is a multi-arm polyether comprising PEG and wherein each polyether arm has from 2 to 150 ethylene oxide repeat units and each polyester arm has from 4 to 200 repeat units; and
ii. a triblock copolymer, wherein the triblock copolymer has the formula:

Av-Bw-Ax
wherein A is a polyester and B is PEG and v and x are the number of repeat units ranging from 1 to 3,000 and w is the number of repeat units ranging from 3 to 300 and v=x or v≠x; and
iii. a diblock copolymer, wherein the diblock copolymer has the formula:

Cy-Az
wherein A is a polyester and C is an end-capped PEG and y and z are the number of repeat units with y ranging from 2 to 250 and z ranging from 1 to 3,000;
iv. or any combination thereof.

3. A pharmaceutical composition according to clause 1 or 2, wherein the composition is liquid at room temperature and forms a semi solid or solid implant when injected into an aqueous environment.

4. A pharmaceutical composition according to any preceding clause wherein the acidic compound d) is an inorganic acid or a carboxylic acid, optionally a polycarboxylic acid, optionally a di or tricarboxylic acid.

5. A pharmaceutical composition according to any preceding clause wherein the acidic compound d) is selected from aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid or tartaric acid or a combination thereof, preferably salicyclic acid, oxalic acid, malonic acid, sulfamic acid, pamoic acid or any combination thereof.

6. A pharmaceutical composition according to any preceding clause, wherein the polyester of the polyether-polyester copolymer a) is poly(D,L-lactic acid) (PLA), poly(D,L-lactic-co-glycolic acid) (PLGA) or poly(ε-caprolactone-co-lactic acid) (PCLA).

7. A pharmaceutical composition according to any of clauses 2 to 6 wherein the end-capped polyethylene glycol is methoxy-polyethylene glycol.

8. A pharmaceutical composition according to any preceding clause wherein the polyester of the polyether-polyester copolymer a) is poly(D,L-lactic acid) (PLA).

9. A pharmaceutical composition according to any preceding clause wherein the polyether-polyester copolymer a) is a multi-arm copolymer i) having a molar ratio of the ester repeat unit to the ethylene oxide repeat unit of from 1 to 10, preferably from 2 to 6.

10. A pharmaceutical composition according to any preceding clause, wherein if the polyether-polyester copolymer a) is a multi-arm copolymer i) the central core is a multi-arm polyether which is obtainable from PEG and a polyol.

11. A composition according to clause 10 wherein the polyol comprises at least three hydroxyl groups, optionally wherein the polyol is a hydrocarbon substituted with at least three hydroxyl groups, optionally 3, 4, 5, 6 or 8 hydroxyl groups.

12. A composition according to clause 10 or clause 11 wherein the polyol is pentaerythritol (PE), dipentaerythritol, trimethylolpropane (TMP), glycerol, erythritol, xylitol, di(trimethylolpropane (diTMP) sorbitol, or inositol.

13. The composition according to any of clauses 10 to 12 wherein the polyol further comprises one or more ether groups.

14. A pharmaceutical composition according to any of clauses 2 to 8, wherein the at least one polyether-polyester copolymer a) is a mixture of a triblock copolymer ii) and a diblock copolymer iii).

15. A pharmaceutical composition according to any of clauses 2 to 8 and 14 wherein the molar ratio of the ester repeat unit to the ethylene oxide repeat unit for the triblock copolymer ii) is from 0.5 to 22, preferably from 0.5 to 10, most preferably from 1 to 6.

16. A pharmaceutical composition according to any of clauses 2 to 8 and 14 wherein the molar ratio of the ester repeat unit to the ethylene oxide repeat unit for the diblock copolymer iii) is from 0.8 to 15, preferably from 1 to 10.

17. A pharmaceutical composition according to any preceding clause, wherein the nucleophilic compound b) comprises one or more functional groups selected from —SH, —OH, —NH₂, —N═H, a tertiary amine, a heterocyclic group and combinations thereof.

18. A pharmaceutical composition according to any preceding clause, wherein the nucleophilic compound b) is an active pharmaceutical ingredient.

19. A pharmaceutical composition according to clause 18 wherein the active pharmaceutical ingredient is selected from the group consisting of octreotide acetate, liothyronine, escitalopram free base, atorvastatin calcium trihydrate or a combination thereof.

20. A pharmaceutical composition according to any of clauses 1 to 17 wherein the nucleophilic compound is not an active pharmaceutical ingredient and wherein the composition further comprises at least one active pharmaceutical ingredient.

21. A pharmaceutical composition according to clause 20 wherein the nucleophilic compound b) is an alcohol, optionally a C₁ to C₈ alcohol, optionally glycerol, sorbitol, methanol, ethanol, propanediol, propylene glycol, polyethylene glycol, preferably methanol, propylene glycol, polyethylene glycol or derivatives or mixtures thereof.

22. A pharmaceutical composition according to any preceding clause wherein the nucleophilic compound b) is a saccharide, disaccharide or polysaccharide, optionally sucrose, dextrose, cyclodextrin, chitosan or mixtures thereof.

23. A pharmaceutical composition according to any preceding clause wherein the nucleophilic compound b) is an amino acid, peptide, or polypeptide, optionally lysine, arginine, histidine or serine.

24. A pharmaceutical composition according to clause 20 wherein the nucleophilic compound b) is water.

25. A pharmaceutical composition according to clause 20 wherein the nucleophilic compound b) is a further organic solvent, optionally pyrrolidone-2, glycofurol, pyridine, nitromethane, triethylamine, N,N-dimethylaniline, N,N-diemthyldecanamide, N,N-dimethyloctanamide, 2,4,6-collidine or mixtures thereof.

26. A pharmaceutical composition according to clause 20 to 25 wherein the nucleophilic compound b) is a solubility enhancer, a porogen or a phase exchange modifier.

27. A pharmaceutical composition according to any preceding clause, wherein the at least one organic solvent c) is selected from the group consisting of benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, triacetin, tributyrin, tripropionin, glycofurol or a mixture thereof, preferably DMSO, NMP and mixtures thereof.

28. A pharmaceutical composition according to any preceding clause, wherein the acidic compound d) has a pKₐ(DMSO) lower than 10, preferably lower than 8.

29. A pharmaceutical composition according to any preceding clause, wherein the amount of the at least one acidic compound d) is from 0.005% (w/w) to 10% (w/w), optionally 0.55% (w/w) to 10% (w/w), or 0.005% (w/w) to 0.45% (w/w), preferably 0.01% (w/w) to 4.0% (w/w) of the total composition.

30. A pharmaceutical composition according to any of preceding clause, wherein the molar amount of the acidic compound d) is 0.05% to 300% relative to the molar amount of the nucleophilic compound b), preferably 0.1% to 250%.

31. A pharmaceutical composition according to any preceding clause, wherein the nucleophilic compound b) contains at least one —OH group and wherein the molar amount of the acidic compound d) is 0.05% to 100% relative to the molar amount of the nucleophilic compound.

32. A pharmaceutical composition according to any preceding clause, wherein the nucleophilic compound b) contains at least one nitrogen containing reactive group such as —NH₂ or ═NH, and wherein the molar amount of the acidic compound d) is greater than 100% relative to the molar amount of the nucleophilic compound, preferably 100% to 300% relative to the amount of the nucleophilic compound.

33. A pharmaceutical composition according to any preceding clause, wherein the total amount of the polyether-polyester copolymer a) is 2% (w/w) to 80% (w/w), optionally 10 to 50% (w/w), optionally 20 to 40% (w/w) of the total composition.

34. A pharmaceutical composition according to any of clauses 2 to 13 or 17 to 33, wherein the polyether-polyester copolymer a) is a multi-arm copolymer i) and the amount of the multi-arm copolymer is from 20 to 60% (w/w), optionally 20 to 50% (w/w) of the total composition.

35. A pharmaceutical composition according to any of clauses 2 to 8 or 14 to 33, wherein the amount of the diblock copolymer is from 2 to 30% (w/w), optionally 10 to 30% (w/w), optionally 10 to 20% (w/w) of the total composition; and the amount of the triblock copolymer is from 2 to 30% (w/w), optionally 10 to 30% (w/w), optionally 10 to 20% (w/w) of the total composition.

36. A pharmaceutical composition according to any of clauses 18 to 35, wherein the amount of the active pharmaceutical ingredient is 0.05% (w/w) to 60% (w/w), optionally 0.05 to 20% (w/w), optionally 0.05 to 10% (w/w), optionally 0.05 to 5% (w/w), optionally 0.05 to 2% (w/w) of the total composition.

37. A pharmaceutical composition according to any preceding clause, wherein the amount of the organic solvent is at least 20% (w/w) of the total composition, optionally 20 to 80% (w/w), optionally 20 to 60% (w/w).

38. A pharmaceutical composition according to any preceding clause, wherein the composition is stable for at least 2 weeks storage at room temperature or 2 to 8° C., preferably at least 4 weeks storage at room temperature or 2 to 8° C.

39. A pharmaceutical composition according to any preceding clause wherein the concentration of the active pharmaceutical ingredient in the composition reduces by less than 20%, preferably less than 10%, more preferably less than 5% after 2 weeks storage at room temperature or 2 to 8° C., preferably 4 weeks storage at room temperature or 2 to 8° C. relative to the initially formulated composition.

40. A pharmaceutical composition according to any preceding clause wherein the dynamic viscosity of the composition reduces by less than 10%, preferably less than 5% after 2 weeks storage at room temperature or 2 to 8° C., preferably 4 weeks storage at room temperature or 2 to 8° C. relative to the initially formulated composition.

41. A method for preparing a pharmaceutical composition as described in any preceding clause consisting of the steps of:
i. dissolving the at least one polyether-polyester copolymer a) as defined in any preceding clause in the at least one organic solvent c), followed by
ii. adding to the product of step i) at least one acidic compound d) as defined in any preceding clause and at least one nucleophilic compound b) as defined in any preceding clause, optionally wherein the nucleophilic compound b) is an active pharmaceutical ingredient followed by
iii. homogenizing the formulation, thereby obtaining the pharmaceutical composition.

42. A method according to clause 41, wherein the nucleophilic compound is not an active pharmaceutical ingredient and step ii) further comprises adding an active pharmaceutical ingredient.

43. A method according to clause 41 or 42, wherein the active pharmaceutical ingredient is previously dissolved in the organic solvent c).

44. A method according to clauses 41 to 43, wherein the acidic compound d) is previously dissolved in the organic solvent c).

45. A method according to any of clauses 41 to 44, wherein the nucleophilic compound b) is previously dissolved in the organic solvent c).

46. A method according to any of clauses 41 to 45 wherein the pharmaceutical composition obtained in step iii. is filtered.

### EXAMPLES

### Example 1: Materials

Copolymers were synthesized according to the method described in the U.S. Pat. No. 6,350,812, incorporated herein by reference, with minor modifications. Typically, the necessary amount of PEG (gives the triblock copolymer) or methoxy-PEG (gives the diblock copolymer) or 4-arm PEG (gives the 4-arm star-shaped copolymer) was heated between 65° C. and dried under vacuum for 2 hours in a reactor vessel. DL-lactide (corresponding to the targeted LA/EO molar ratio) and catalyst (such as 1/1000 of amount of lactide) were added. The reaction mixture was first dehydrated by several short vacuum/N₂ cycles. The reaction mixture was heated at 140° C. and rapidly degassed under vacuum. The reaction was conducted for several hours at 140° C. under constant nitrogen flow (0.2 bar). The reaction was cooled to room temperature and its content was dissolved in acetone and then subjected to precipitation with ethanol. The product obtained was subsequently dried under reduced pressure.

The triblock PLA-PEG-PLA polymers described herein are labelled PxRy, where x represent the size of the PEG chain in kDa (number average molecular weight) and y is the LA/EO molar ratio. The diblock mPEG-PLA polymers described herein are labelled dPxRy where x represents the size of the PEG chain in kDa (number average molecular weight) and y is the LA/EO molar ratio. The star-shaped sPEG-PLA polymers described herein are labelled szPxRy where x represents the size of the PEG chain in kDa (number average molecular weight), y is the LA/EO molar ratio and z the arm number.

The product obtained was characterized by ¹H NMR for its residual lactide content and for the determination of the R ratio. ¹H NMR spectroscopy was performed using a Brucker Advance 300 MHz spectrometer. For all ¹H NMR spectrograms, topspin software was used for the integration of peaks and their analyses. Chemical shifts were referenced to the δ=7.26 ppm solvent value of CDCl₃.

For the determination of the R ratio, which describes the ratio between lactic acid units over ethylene oxide units (LA/EO), all peaks were integrated separately. The intensity of the signal (integration value) is directly proportional to the number of hydrogens that constitutes the signal. To determine the R ratio (LA/EO ratio), the integration values need to be homogenous and representative of the same number of protons (e.g. all signal values are determined for ¹H). One characteristic peak of PLA and one of PEG are then used to determine the LA/EO ratio. This method is valid for molecular weights of PEGs above 1000 g/mol where the signal obtained for the polymer end-functions can be neglected.

### Example 2: Vehicles and Formulations Preparation

Typically, vehicles (pharmaceutical composition in the absence of API) were prepared by adding DMSO using a Pasteur pipette on top of copolymers previously weighed. The mixture was stirred on roller mixer at RT until a homogenous solution was obtained.

For formulations, API was weighed in another empty and tarred glass vial. 30 min before the beginning of the experiment, the required amount of vehicle was added on top it. The vial was vortexed for around 30 s and placed on a roller mixer at RT until the 1^{st} analysis.

When needed, additional excipients (alcohols or acids) were added the day of study start directly into the vehicle vial before formulation reconstitution or by first dissolving them in a solution containing DMSO.

In the case of liothyronine based formulations, vials were nitrogen-flushed before being placed on the roller mixer.

In the case of atorvastatin based formulations, due to the higher API contents, tested formulations were vortexed for 1 min after vehicle addition and a longer homogenisation time of around 1 hour on roller mixer was required.

Tested acids are listed in table 1.

TABLE 1

| Name | Structure | Mw (g/mol) | pKₐ(H₂O) | pKₐ(DMSO) |
|---|---|---|---|---|
| Formic acid | 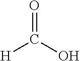 | 46 | 3.75 | — |
| Lactic acid | 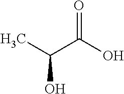 | 90 | 3.86 | — |
| Oxalic acid | 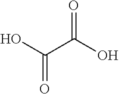 | 90 | 1.46 | 6.2 |
| Sulfamic acid | 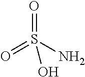 | 97 | 0.99 | 6.5 |
| Malonic acid | 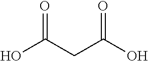 | 104 | 2.85 | 10.6 |
| Benzoic acid | 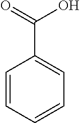 | 122 | 4.19 | 11.1 |
| Salicylic acid | 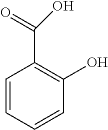 | 138 | 2.79 | 6.8 |
| Pamoic acid | 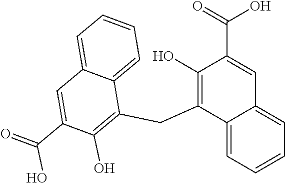 [SMILES: O=C(O)c1cc2ccccc2c(Cc2c(O)c(C(=O)O)cc3ccccc23)c1O] | 388 | 2.67 | — |

“—”: Not found in the literature

### Example 3: Octreotide Formulations Degradation and Stability Studies

Impact of acid addition within octreotide acetate formulations was assessed through degradation and stability studies detailed in table 2.

TABLE 2

| Degradation study | | | Stability study | | |
|---|---|---|---|---|---|
| Timepoints | Temperature | Analyses | Timepoints | Temperature | Analyses |
| t0; t4w; t8w | 40° C. | Assay, | 0.5; 2.5; 4; 6; 24; | RT | Assay, visual |
| | | rheology, visual | 48 and 240 h | | observations |
| | | observations | t0; t2w; t4w | 4° C. | Assay, rheology, |
| | | | | | visual observations, |
| | | | | | IVR |

Test items were prepared as described in example 2. For studies longer than 10 days, vehicles and formulations were further aliquoted according to the number of timepoints.

Detailed descriptions of analyses are given below.

### API Content Determination

Drug content determinations were performed on formulations 30 min after vehicles addition and at different pre-determined timepoints as disclosed in table 2.

Assays were performed as described below:
Around 120 μL of formulation was withdrawn into a 0.5 mL syringe with a 23 G*1″ needle, and all air bubbles were removed.
Syringe containing the formulation was placed on the balance and the balance was tarred.
40 μL of formulation were injected into a labelled 50 mL empty falcon tube.
Syringe was weighed back, and the exact formulation mass injected in the falcon tube was recorded.
Formulation was dissolved in 4 mL of HPLC-grade acetonitrile and the solution was vortexed until complete dissolution.
16 mL of H₂O+0.1% TFA was further added in each Falcon tube.
Solution was vortexed until complete homogenization.
1 mL of each sample was withdrawn and put into a 1.5 mL Eppendorf tube.
Eppendorf tube was centrifugated for 5 min at 3,500 rpm and 800 μL of clear supernatant were then transferred in a 1 mL HPLC glass vial.
Back-up samples were stored at +4° C. until the end of the experiment.

API content was determined using the appropriate LC method. Drug content analyses were performed in triplicate. Results are expressed as a recovery % and takes as reference the experimental drug content calculated from masses weighed during formulation preparation.

### Visual Observation:

Vehicles and formulations were visually observed at each timepoint and compared with coloration standards.

### Viscosity Analyses:

Dynamic viscosity was determined using an Anton Paar Rheometer equipped with a cone plate measuring system with a diameter of 50 mm and a cone angle of 1 degree. After being vortexed, formulations were placed at the center of the thermo-regulated measuring Peltier plate. The measuring system was lowered down and a 0.104 mm gap was left between the measuring system and the measuring plate. Twenty-one viscosity measurement points were then determined across the 10 to 1,000 s⁻¹ shear rate range. Given viscosity data refers to that calculated at a shear rate of 100 s⁻¹, corresponding to an average value of the curve plateau. Analyses were performed on triplicate or duplicate.

### IVR:

100 μL of test items were withdrawn from the corresponding glass vial previously vortexed, into a 0.5 mL Codan syringe with a 18 G needle. The syringe was cleaned, tared, needle removed and formulation was directly injected into a vial prefilled with 20 mL of KRT-1X. Once polymer precipitation had occurred, depots were separated from the syringe and the syringe was weighed back. Sample mass was recorded. IVR tests were performed in triplicate and once all depots were formed, glass vials were placed on a stirrer at 37° C.

At each desired time point, around 2 mL of buffer were withdrawn from the glass vial before total buffer refreshment. Samples were filtered through a 0.2 μm hydrophilic into a 1.5 mL HPLC glass vial and then analyzed using the appropriate LC method.

Table 3 discloses the compositions of tested octreotide acetate formulations.

TABLE 3

| | API % | P1R4 % | dP2R3 % | DMSO % | | Coexcipient % | Acid/Octreo |
|---|---|---|---|---|---|---|---|
| Formulation | (w/w) | (w/w) | (w/w) | (w/w) | Coexcipient | (w/w) | molar ratio |
| F17 | 4.3 | 4.0 | 16.0 | 64.2 | Pamoic acid | 1.5 | 1.00 |
| | | | | | Propylene glycol | 10.0 | NA |
| F18 | 4.3 | 4.0 | 16.0 | 60.7 | Pamoic acid | 5.0 | 3.28 |
| | | | | | Propylene glycol | 10.0 | NA |
| F19 | 4.0 | 10.0 | 10.0 | 76.0 | — | — | NA |
| F20 | 4.0 | 10.0 | 10.0 | 74.5 | Pamoic acid | 1.5 | 1.08 |
| F21 | 4.0 | 10.0 | 10.0 | 72.0 | Pamoic acid | 4.0 | 2.88 |
| F22 | 4.0 | 20.0 | 20.0 | 56.0 | — | — | NA |
| F23 | 4.0 | 20.0 | 20.0 | 54.5 | Pamoic acid | 1.5 | 1.08 |
| F24 | 4.0 | 20.0 | 20.0 | 52.0 | Pamoic acid | 4.0 | 2.88 |
| F25 | 4.3 | 4.0 | 16.0 | 74.2 | Pamoic acid | 1.5 | 1.00 |
| F26 | 4.3 | 4.0 | 16.0 | 70.7 | Pamoic acid | 5.0 | 3.28 |
| F30 | 4.3 | 20.0 | 20.0 | 54.4 | SDS | 1.1 | NA |
| F31 | 4.3 | 20.0 | 20.0 | 53.8 | Docusate | 1.9 | NA |
| F32 | 4.3 | 20.0 | 20.0 | 55.1 | Salicylic acid | 0.6 | 1.00 |
| F33 | 4.3 | 20.0 | 20.0 | 55.5 | Formic acid | 0.2 | 1.00 |
| F34 | 4.3 | 20.0 | 20.0 | 52.1 | SAIB | 3.6 | NA |
| F35 | 4.3 | 20.0 | 20.0 | 54.7 | BHT | 0.9 | NA |
| F37 | 4.3 | 20.0 | 20.0 | 55.3 | Oxalic acid | 0.4 | 1.00 |
| F38 | 4.3 | 20.0 | 20.0 | 55.2 | Benzoic acid | 0.5 | 1.00 |
| F39 | 4.3 | 4.0 | 16.0 | 75.7 | — | — | NA |
| F49 | 4.3 | 20.0 | 20.0 | 54.7 | Pamoic acid | 1.0 | 0.67 |
| F50 | 4.3 | 20.0 | 20.0 | 54.7 | Oxalic acid | 1.0 | 2.50 |
| F51 | 4.3 | 20.0 | 20.0 | 54.7 | Formic acid | 1.0 | 5.00 |
| F52 | 4.3 | 20.0 | 20.0 | 53.6 | Pamoate disodium | 2.1 | NA |
| | | | | | salt | | |
| F53 | 4.3 | 20.0 | 20.0 | 55.3 | Sulfamic acid | 0.4 | 1.00 |
| F122 | 4.3 | 20.0 | 20.0 | 54.90 | Oxalic acid | 0.57 | 1.50 |
| F123 | 4.3 | 20.0 | 20.0 | 55.67 | — | — | NA |
| F124 | 4.3 | 20.0 | 20.0 | 55.09 | Oxalic acid | 0.77 | 2.00 |

NA: Not applicable

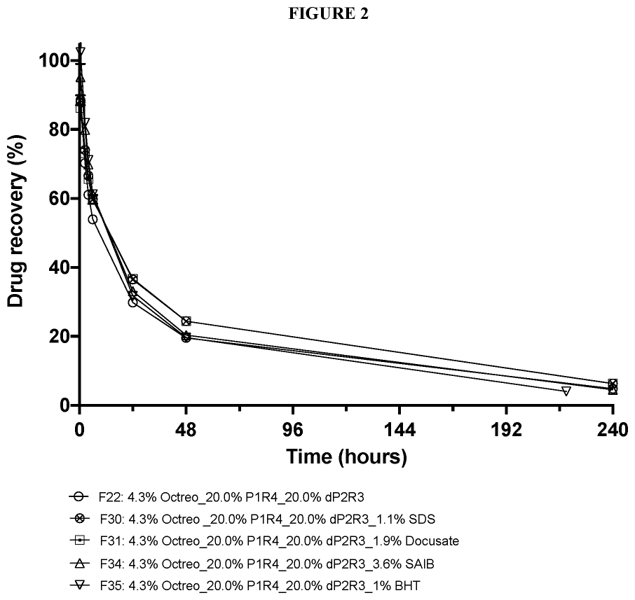

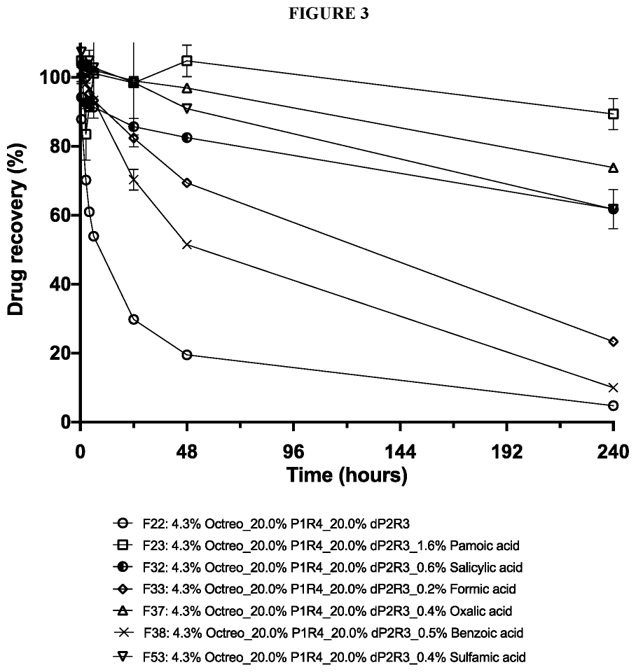

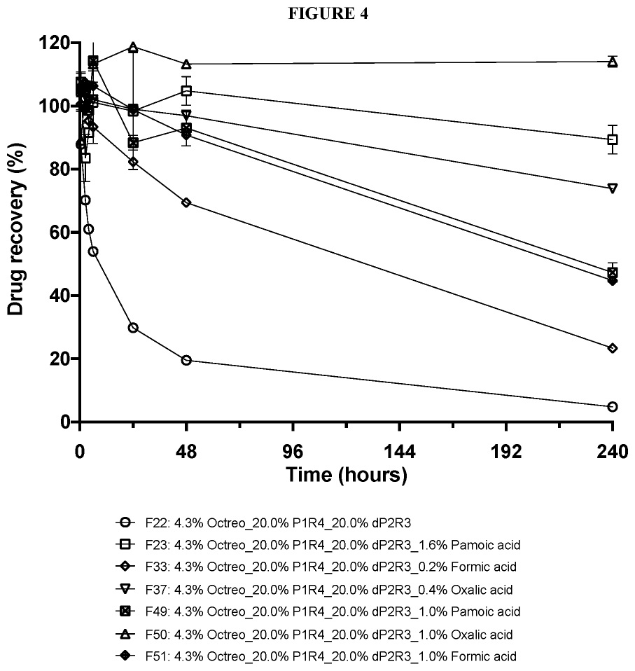

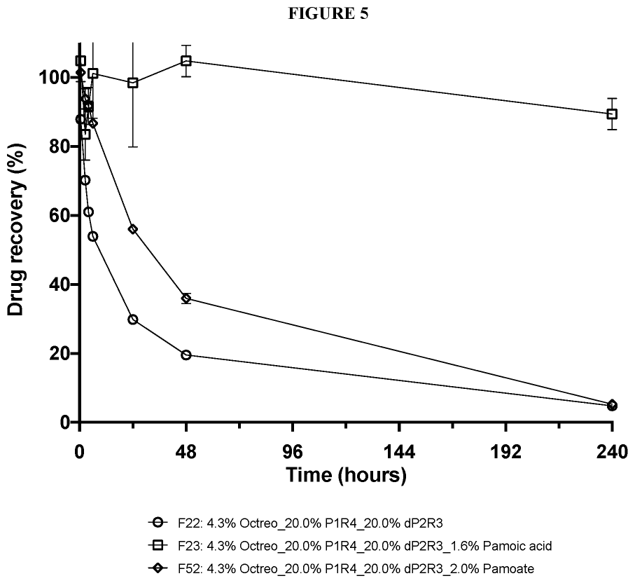

FIGS. 2 to 5 present drug recovery over time of tested formulations after 10 days at room temperature (RT). Data show that only the addition of acids allowed to efficiently reduce the peptide acylation over time. In particular, FIG. 2 presents the results obtained with other co-excipients, for which peptide recoveries are similar to the control formulation. As shown in FIG. 3 at a fixed equimolar acid/peptide ratio, the acid pKₐ in water has an impact on drug recovery over time. Acids with pKₐ(H₂O) higher than 3 (benzoic and formic acids) achieved poor peptide recovery. Moreover, as illustrated in FIG. 4 the acid concentration within formulations also has an impact on the drug recovery level: with tested concentrations, the higher the acid loading within formulation, the higher the peptide recovery. As illustrated in FIG. 5, the protonation state of the co-excipient is key, as no improvement in drug recovery was observed with the formulation containing the pamoate salt.

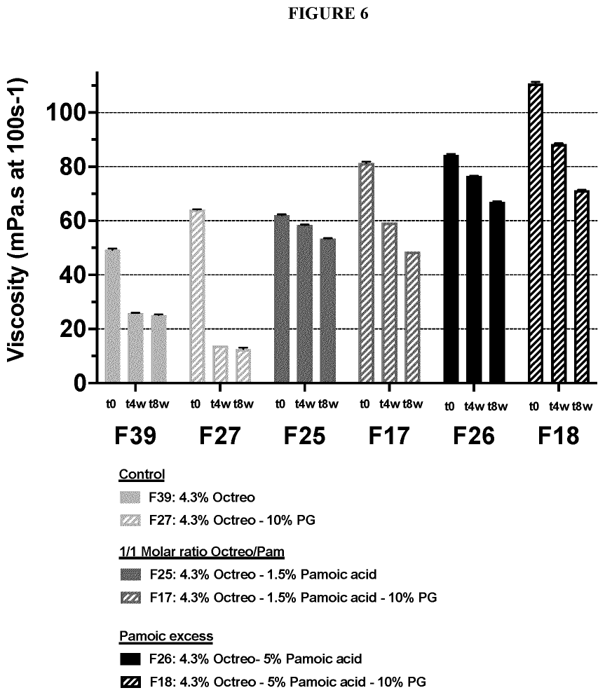

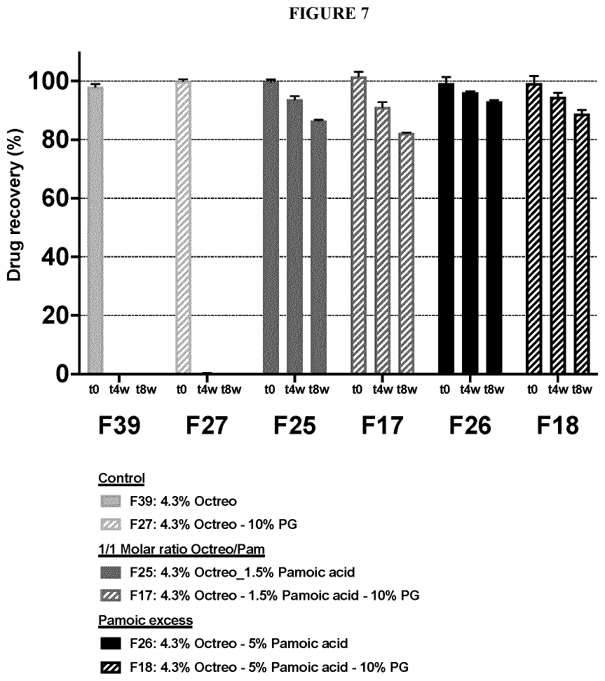

Results of the 2-month forced degradation at 40° C. study are disclosed in FIGS. 6 and 7. While no native peptide was detected after 1 month in control formulations with no acid, all formulations containing pamoic acid presented a drug recovery higher than 80% at the end of the study. In the simultaneous presence of 2 nucleophiles (propylene glycol and octreotide), pamoic acid also highly improves formulation stability. The higher degradation of control formulations is confirmed by their viscosity reductions.

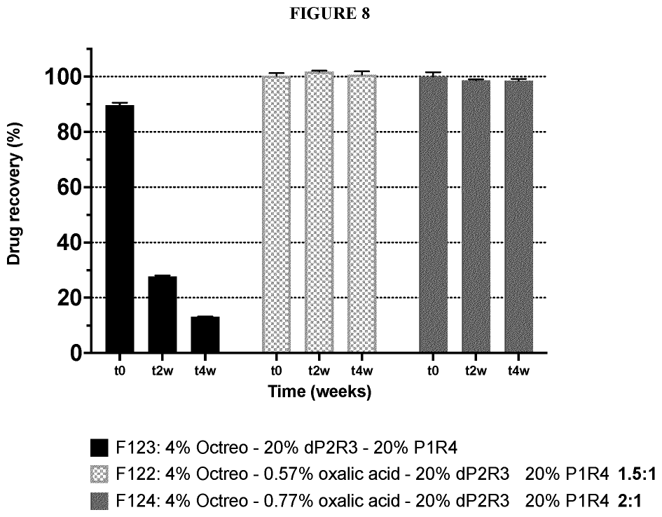

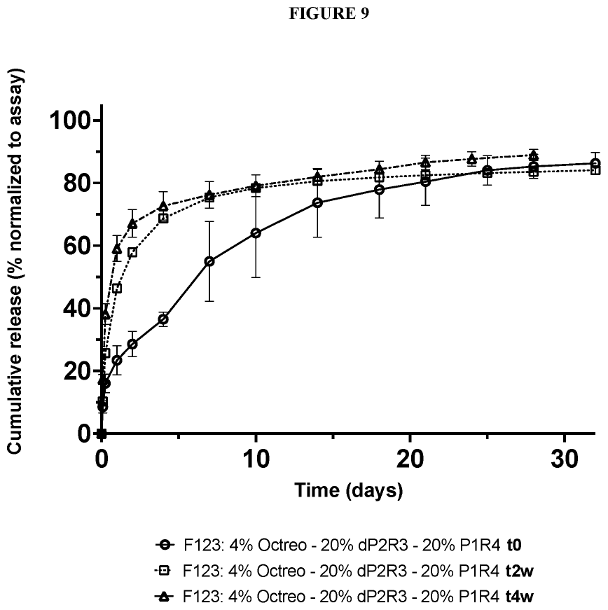

FIGS. 8 and 9 present the results of the 4-week stability study at 4° C. No decrease in drug recoveries can be observed at the end of the study for formulations F122 and F124 containing oxalic acid. Less than 20% of native peptide are recovered in control formulation F123 with no acid. As for the 2-month degradation study, this is in accordance with the viscosity of control formulation decreasing with time and with a decrease of native peptide cumulative release as shown on FIG. 9. While the formulations containing oxalic acid present reproducible release profiles at study start or after 2 and 4 weeks at 4° C., the percentage of native peptide cumulative released from the control formulation decreases after 2 and 4 weeks of storage.

### Example 4: Stability Studies of Liothyronine Formulations

The impact of acid addition on liothyronine formulations was also evaluated. Drug content analyses were performed as disclosed in example 3 with minor modifications: 200 mg samples were dissolved in a 20 mL volumetric flask with a ACN/H₂O 80/20 mixture.

Table 4 discloses the compositions of tested Lyothyronine formulations.

TABLE 4

| | API % | TB | | dP2R3 % | DMSO % | | Coexcipient % | Oxalic/Lio |
|---|---|---|---|---|---|---|---|---|
| Formulation | (w/w) | type | % (w/w) | (w/w) | (w/w) | Coexcipient | (w/w) | molar ratio |
| F32 | 1.00 | P2R2 | 10.00 | 10.00 | 79.00 | — | — | NA |
| F39 | 0.20 | P2R2 | 10.00 | 10.00 | 79.80 | — | — | NA |
| F46 | 1.00 | P2R2 | 10.00 | 10.00 | 78.00 | Oxalic acid | 1.00 | 8/1 |
| F47 | 1.00 | P2R2 | 10.00 | 10.00 | 78.00 | Pamoic acid | 1.00 | NA |
| F48 | 1.00 | P2R2 | 10.00 | 10.00 | 78.00 | CaCl₂ | 1.00 | NA |
| F50 | 0.20 | P2R2 | 10.00 | 10.00 | 79.7975 | Oxalic acid | 0.0025 | 10/100 |
| F51 | 0.20 | P2R2 | 10.00 | 10.00 | 79.795 | Oxalic acid | 0.005 | 20/100 |
| F52 | 0.20 | P2R2 | 10.00 | 10.00 | 79.775 | Oxalic acid | 0.025 | 1/1 |
| F53 | 0.20 | P2R2 | 10.00 | 10.00 | 79.75 | Oxalic acid | 0.05 | 2/1 |
| F54 | 0.20 | P2R2 | 10.00 | 10.00 | 79.70 | Oxalic acid | 0.10 | 4/1 |
| F55 | 0.20 | P2R2 | 10.00 | 10.00 | 79.55 | Oxalic acid | 0.25 | 10/1 |
| F56 | 0.20 | P2R2 | 10.00 | 10.00 | 79.30 | Oxalic acid | 0.50 | 20/1 |
| F57 | 0.20 | P1R4 | 10.00 | 10.00 | 79.80 | — | — | NA |
| F58 | 0.20 | P1R4 | 10.00 | 10.00 | 79.70 | Oxalic acid | 0.10 | 4/1 |
| F59 | 0.20 | P1R4 | 10.00 | 10.00 | 79.55 | Oxallic acid | 0.25 | 10/1 |

NA: Not applicable

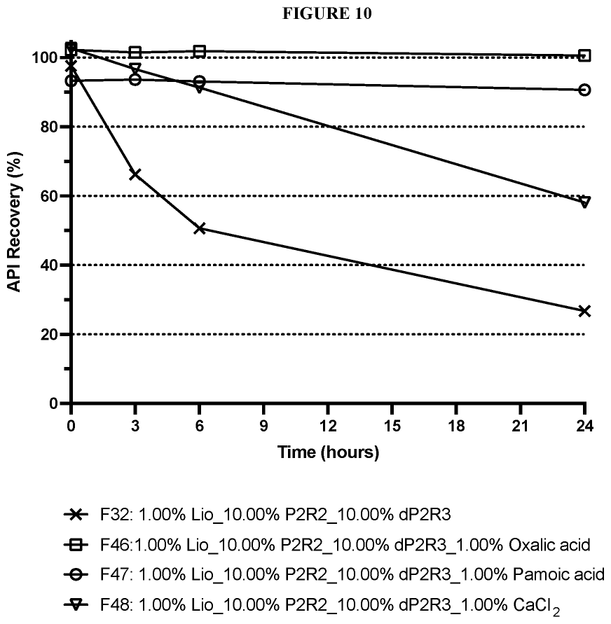

A first test was launched at RT to compare the impact of different coexcipients on API recovery after formulation reconstitution and 3, 6 and 24 h later. Coexcipient and Liothyronine contents were fixed at 1% (w/w). Assays were performed in duplicate. Results are presented in FIG. 10. As for octreotide formulations, the addition of pamoic or oxalic acids resulted in higher drug recoveries over time compared to control formulation or to formulation F48 containing CaCl₂), a divalent cation commonly used to reduce molecule acylation.

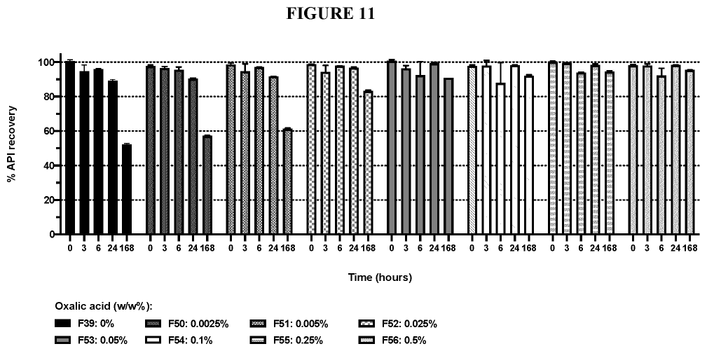

The impact of oxalic acid content was then further evaluated over 7 days on formulations loaded with 0.2% of Liotyronine. Results are presented in FIG. 11. In presence of 0.005 to 0.50% oxalic acid, higher Liothyronine contents were measured with time. In particular, with formulations F55 and F56, containing 0.25 and 0.50% oxalic acid, over 95% of API were recovered at all timepoints.

A 2-week short-term stability study at RT and 4° C. was launched with formulations containing 0.10 and 0.25% oxalic acid. Drug content, rheology and visual observations were performed on selected formulations at study start (t0) and after 3; 7 and 14 days (t3D; t7D and t14D) as disclosed in example 3 and above. Drug contents were performed in triplicate and rheology analyses in duplicate. Drug content results are expressed as a recovery % and takes as reference the drug content calculated measured at study start.

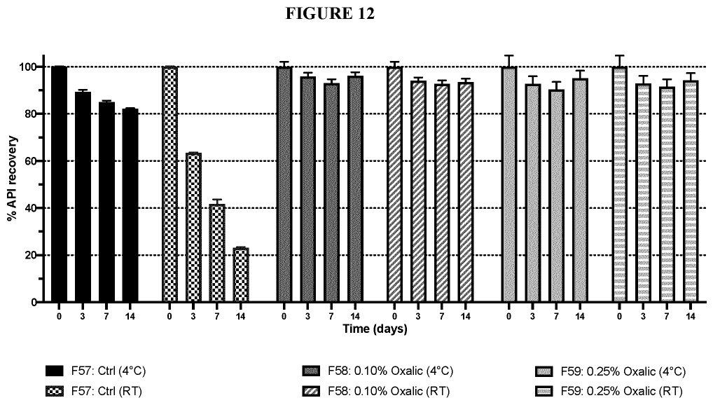

The viscosity values of all formulations were stable throughout the study, with less than 5% variation from initial value at all time points. A coloration of control formulation F57 with no oxalic acid was noted after 3 days at RT. However, in presence of acid or when stored at 4° C., no coloration were observed. FIG. 12 presents the drug recoveries obtained after up to 2 weeks at RT or 4° C. While close to 95% of the initial API content were recovered from formulations F58 and F59 containing oxalic acid stored at RT or 4° C., a decrease in drug recovery is noticed with time in control formulation F57. Despite improvements when stored at 4° C., almost 20% of F57 initial API dose was not recovered after 2 weeks. No differences between the two acid contents tested, 0.10 and 0.25%, were observed.

### Example 5: Degradation Study of Vehicle Containing Alcohols

The impact of acid addition on vehicles containing alcohols was assessed through 4-week degradation studies at 50° C. Vehicles appearance and rheology were determined at study start (t0) and after 2 and 4 weeks (t2w and t4w) as described in example 3.

Table 5 presents the compositions of tested vehicles with alcohols and their respective controls.

TABLE 5

| | P1R4 % | dP2R3 % | s4P2R4 % | DMSO % | | Alcohol % | | Acid % | Acid/OH |
|---|---|---|---|---|---|---|---|---|---|
| Vehicle | (w/w) | (w/w) | (w/w) | (w/w) | Alcohol | (w/w) | Acid | (w/w) | molar ratio |
| V54 | 10.00 | 10.00 | — | 80.00 | — | — | — | — | NA |
| V55 | 20.00 | 20.00 | — | 60.00 | — | — | — | — | NA |
| V56 | — | — | 40.00 | 60.00 | — | — | — | — | NA |
| V57 | 10.00 | 10.00 | — | 70.00 | PG | 10.00 | — | — | NA |
| V58 | 20.00 | 20.00 | — | 50.00 | PG | 10.00 | — | — | NA |
| V59 | — | — | 40.00 | 50.00 | PG | 10.00 | — | — | NA |
| V60 | 10.00 | 10.00 | NA | 79.00 | — | — | Pamoic acid | 1.00 | NA |
| V61 | 20.00 | 20.00 | NA | 59.00 | — | — | Pamoic acid | 1.00 | NA |
| V62 | — | — | 40.00 | 59.00 | — | — | Pamoic acid | 1.00 | NA |
| V63 | 10.00 | 10.00 | — | 69.00 | PG | 10.00 | Pamoic acid | 1.00 | 2/100 |
| V64 | 20.00 | 20.00 | — | 49.00 | PG | 10.00 | Pamoic acid | 1.00 | 2/100 |
| V65 | — | — | 40.00 | 49.00 | PG | 10.00 | Pamoic acid | 1.00 | 2/100 |
| V66 | 20.00 | 20.00 | — | 59.49 | — | — | Pamoic acid | 0.51 | NA |
| V67 | 20.00 | 20.00 | — | 54.90 | — | — | Pamoic acid | 5.10 | NA |
| V68 | 20.00 | 20.00 | — | 59.88 | — | — | Oxalic acid | 0.12 | NA |
| V69 | 20.00 | 20.00 | — | 59.76 | — | — | Oxalic acid | 0.24 | NA |
| V70 | 20.00 | 20.00 | — | 58.82 | — | — | Oxalic acid | 1.18 | NA |
| V71 | 20.00 | 20.00 | — | 49.49 | PG | 10.00 | Pamoic acid | 0.51 | 1/100 |
| V72 | 20.00 | 20.00 | — | 44.90 | PG | 10.00 | Pamoic acid | 5.10 | 10/100 |
| V73 | 20.00 | 20.00 | — | 49.88 | PG | 10.00 | Oxalic acid | 0.12 | 1/100 |
| V74 | 20.00 | 20.00 | — | 49.76 | PG | 10.00 | Oxalic acid | 0.24 | 2/100 |
| V75 | 20.00 | 20.00 | — | 48.82 | PG | 10.00 | Oxalic acid | 1.18 | 10/100 |
| V76 | 20.00 | 20.00 | — | 59.84 | — | — | Benzoic acid | 0.16 | NA |
| V77 | 20.00 | 20.00 | — | 59.82 | — | — | Salicylic | 0.18 | NA |
| | | | | | | | acid | | |
| V78 | 20.00 | 20.00 | — | 59.86 | — | — | Malonic acid | 0.14 | NA |
| V79 | 20.00 | 20.00 | — | 59.88 | — | — | Lactic acid | 0.12 | NA |
| V80 | 20.00 | 20.00 | — | 59.87 | — | — | Sulfamic | 0.13 | NA |
| | | | | | | | acid | | |
| V81 | 20.00 | 20.00 | — | 49.84 | PG | 10.00 | Benzoic acid | 0.16 | 1/100 |
| V82 | 20.00 | 20.00 | — | 49.82 | PG | 10.00 | Salicylic | 0.18 | 1/100 |
| | | | | | | | acid | | |
| V83 | 20.00 | 20.00 | — | 49.86 | PG | 10.00 | Malonic acid | 0.14 | 1/100 |
| V84 | 20.00 | 20.00 | — | 49.88 | PG | 10.00 | Lactic acid | 0.12 | 1/100 |
| V85 | 20.00 | 20.00 | — | 49.87 | PG | 10.00 | Sulfamic | 0.13 | 1/100 |
| | | | | | | | acid | | |
| V86 | 20.00 | 20.00 | — | 59.74 | — | — | Pamoic acid | 0.26 | NA |
| V87 | 20.00 | 20.00 | — | 59.95 | — | — | Pamoic acid | 0.05 | NA |
| V88 | 20.00 | 20.00 | — | 59.94 | — | — | Oxalic acid | 0.06 | NA |
| V89 | 20.00 | 20.00 | — | 59.99 | — | — | Oxalic acid | 0.01 | NA |
| V90 | 20.00 | 20.00 | — | 59.91 | — | — | Salicylic | 0.09 | NA |
| | | | | | | | acid | | |
| V91 | 20.00 | 20.00 | — | 59.98 | — | — | Salicylic | 0.02 | NA |
| | | | | | | | acid | | |
| V92 | 20.00 | 20.00 | — | 49.74 | PG | 10.00 | Pamoic acid | 0.26 | 0.5/100 |
| V93 | 20.00 | 20.00 | — | 49.95 | PG | 10.00 | Pamoic acid | 0.05 | 0.1/100 |
| V94 | 20.00 | 20.00 | — | 49.94 | PG | 10.00 | Oxalic acid | 0.06 | 0.5/100 |
| V95 | 20.00 | 20.00 | — | 49.99 | PG | 10.00 | Oxalic acid | 0.01 | 0.1/100 |
| V96 | 20.00 | 20.00 | — | 49.91 | PG | 10.00 | Salicylic | 0.09 | 0.5/100 |
| | | | | | | | acid | | |
| V97 | 20.00 | 20.00 | — | 49.98 | PG | 10.00 | Salicylic | 0.02 | 0.1/100 |
| | | | | | | | acid | | |
| V103 | 20.00 | 20.00 | — | 50.00 | PEG1000 | 10.00 | — | — | NA |
| V104 | 20.00 | 20.00 | — | 50.00 | MeOH | 10.00 | — | — | NA |
| V105 | 20.00 | 20.00 | — | 49.999 | PEG1000 | 10.00 | Oxalic acid | 0.001 | 0.1/100 |
| V106 | 20.00 | 20.00 | — | 49.99 | PEG1000 | 10.00 | Oxalic acid | 0.01 | 1/100 |
| V107 | 20.00 | 20.00 | — | 49.95 | PEG1000 | 10.00 | Oxalic acid | 0.05 | 5/100 |
| V108 | 20.00 | 20.00 | — | 49.97 | MeOH | 10.00 | Oxalic acid | 0.03 | 0.1/100 |
| V109 | 20.00 | 20.00 | — | 49.72 | MeOH | 10.00 | Oxalic acid | 0.28 | 1/100 |
| V110 | 20.00 | 20.00 | — | 49.60 | MeOH | 10.00 | Oxalic acid | 1.40 | 5/100 |

NA: Not applicable

No coloration of tested vehicles was observed.

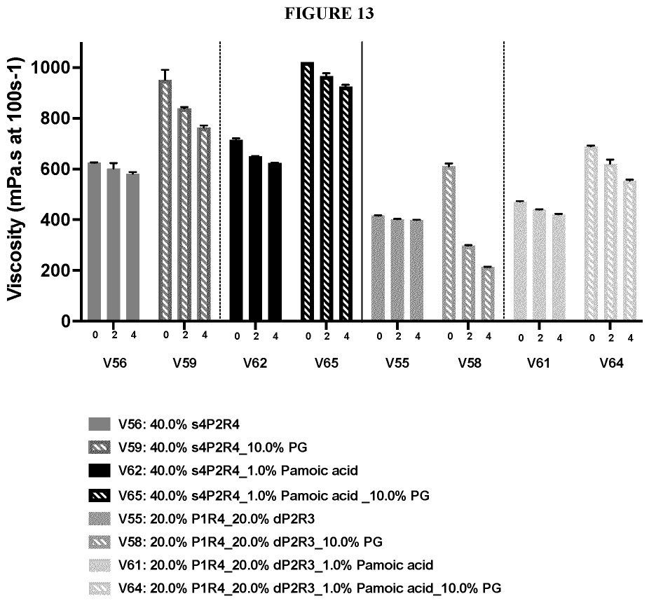

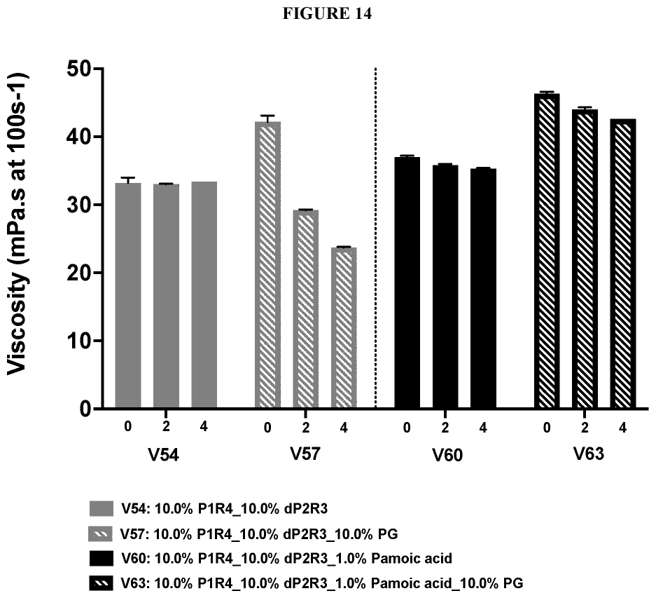

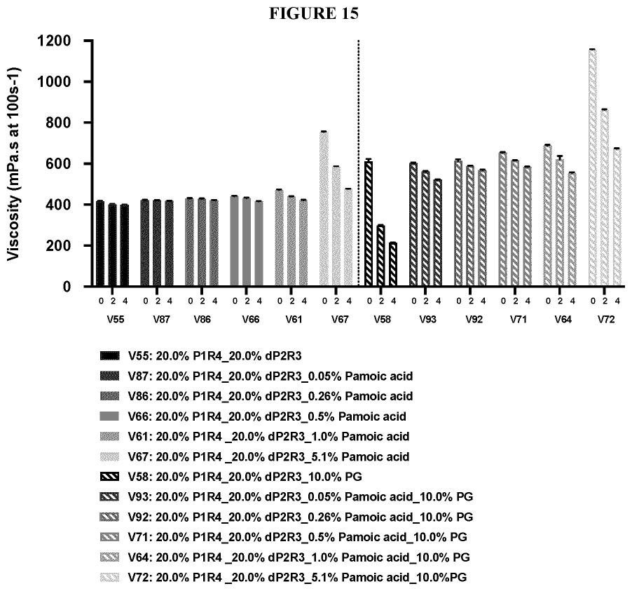

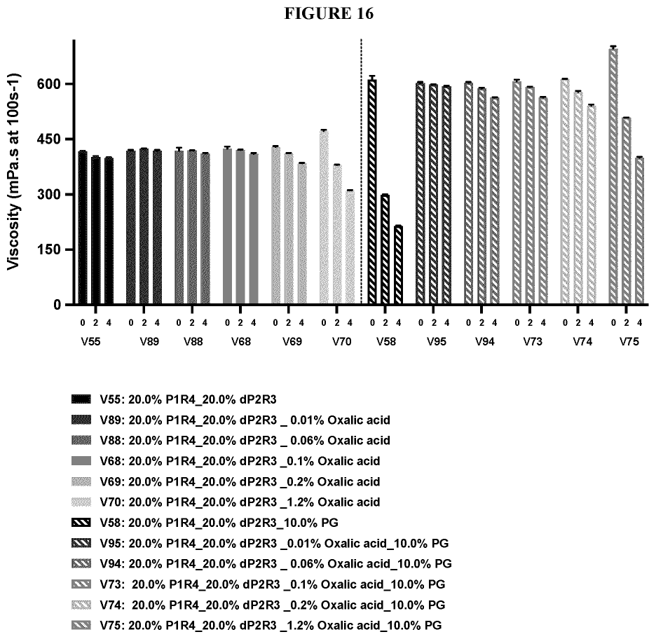

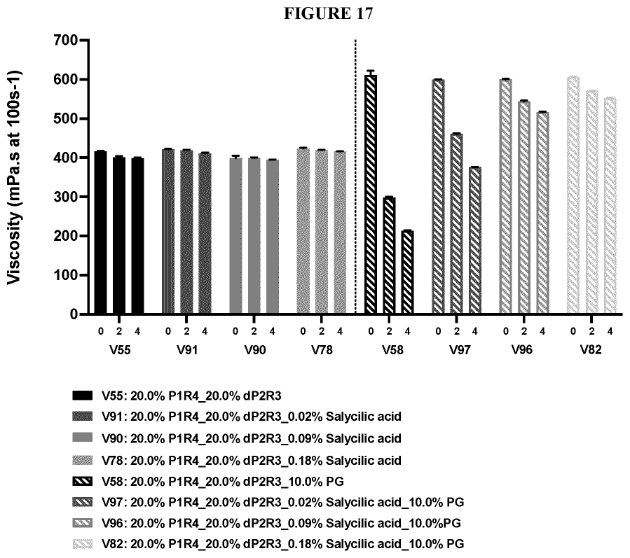

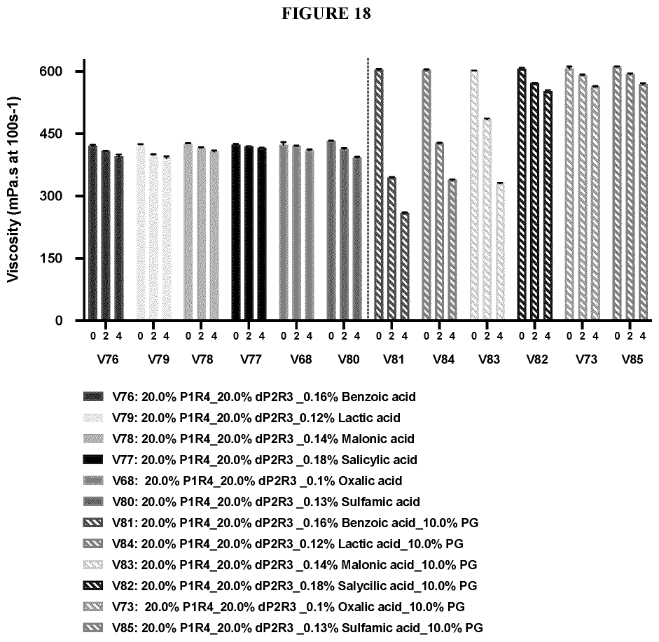

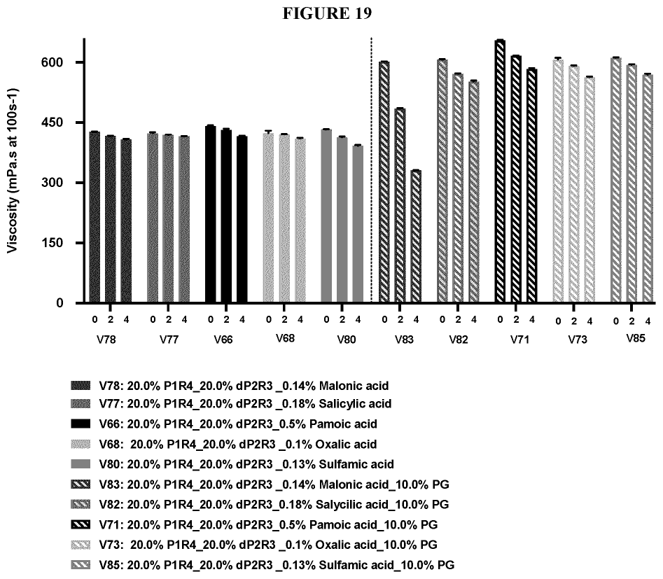

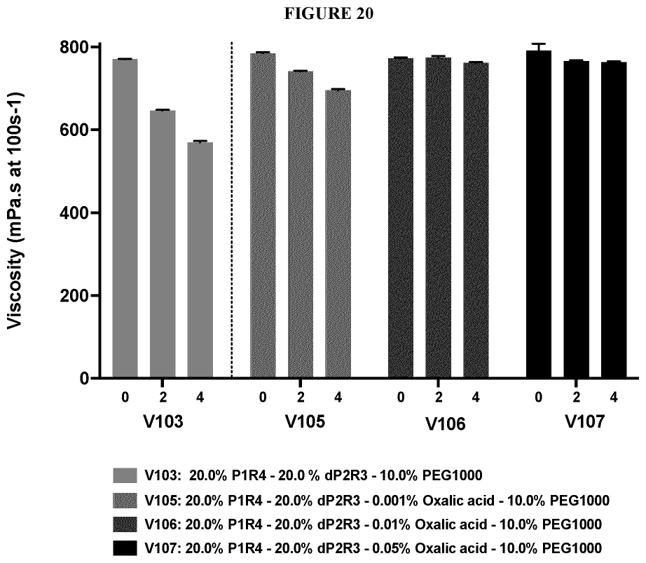

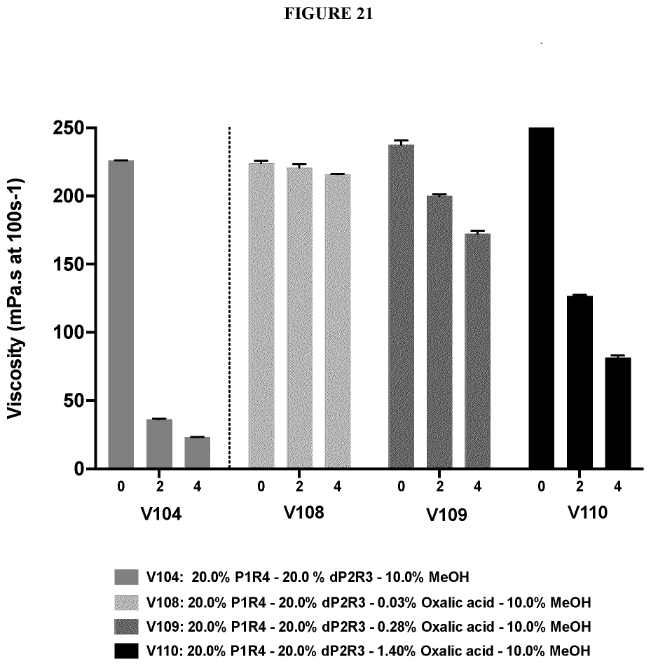

FIGS. 13 to 21 present the results obtained from rheology analyses. It can be seen that the addition of alcohol has a strong impact on vehicle viscosity and thus on polymer stability. However, with the addition of acid, this viscosity decrease is limited despite acid/alcohol molar ratios being equal or lower than 5/100. Very low amounts of acids, such as 0.01% (w/w %) of oxalic acid, efficiently reduced the viscosity decrease induced by the alcohol. In order to achieve a substantial protection against degradation, the amount of acid must be adjusted depending on the alcohol, as illustrated with PEG1000 and methanol in FIGS. 20 and 21. FIG. 18 in particular illustrates the influence of acid characteristics: when comparing acids of similar molecular weight but different pKₐ(H₂O), it can be concluded that the lower the pKₐ(H₂O), the lower the polymer degradation. More precisely, the pKₐ(DMSO) seems to be the parameter leading the degradation reduction as seen on FIG. 19 where salycilic, pamoic, oxalic and sulfamic acids present similar results despite pKₐ (H₂O) varying from 2.79 to 0.99.

### Example 6: Degradation and Stability Studies of Escitalopram Formulations

Forced degradation and stability studies were performed on escitalopram free base or escitalopram oxalate formulations, as detailed in table 6 and according to example 3 with minor modifications. Drug contents were determined from samples dissolved in 20 mL of a 70/30 ACN/H₂O mixture and in vitro depots were formed in gelatin caspules size-00 before being transferred into a vial prefilled with 20 mL of PBS-1X.

TABLE 6

| Degradation study | | | Stability study | | |
|---|---|---|---|---|---|
| Timepoints | Temperature | Analyses | Timepoints | Temperature | Analyses |
| t0; t1w; t2w | 80° C. | Assay, rheology, | t0; t2w; t4w | RT, 4° C.* | Assay, rheology, visual |
| | | visual observations | | | observations, IVR |

*only for the t4w timepoint

Table 7 discloses the compositions of tested Escitalopram formulations.

TABLE 7

| | | API % | P1R4 % | dP2R3 % | DMSO % | Oxalic acid % | Oxalic/Esc |
|---|---|---|---|---|---|---|---|
| Formulation | API | (w/w) | (w/w) | (w/w) | (w/w) | (w/w) | molar ratio |
| F111 | Esc-Base | 5.00 | 15.00 | 15.00 | 65.00 | — | NA |
| F112 | Esc-Ox | 6.40 | 15.00 | 15.00 | 63.60 | — | NA |
| F114 | Esc-Base | 5.00 | 15.00 | 15.00 | 64.999 | 0.001 | 0.1/100 |
| F115 | Esc-Base | 5.00 | 15.00 | 15.00 | 64.99 | 0.01 | 1/100 |
| F116 | Esc-Base | 5.00 | 15.00 | 15.00 | 63.31 | 1.39 | 1/1 |
| F117 | Esc-Base | 5.00 | 15.00 | 15.00 | 62.22 | 2.78 | 2/1 |
| F118 | Esc-Base | 5.00 | 15.00 | 15.00 | 62.92 | 2.08 | 1.5/1 |
| F119 | Esc-Base | 5.00 | 15.00 | 15.00 | 64.31 | 0.69 | 0.5/1 |

NA: Not applicable

While a strong coloration was observed when compared to the control escitalopram free base formulation F111 after 2 weeks at 80° C. or 4 weeks at RT, the addition of an excess of oxalic acid (F117 and F118) resulted in a reduction of formulation coloration.

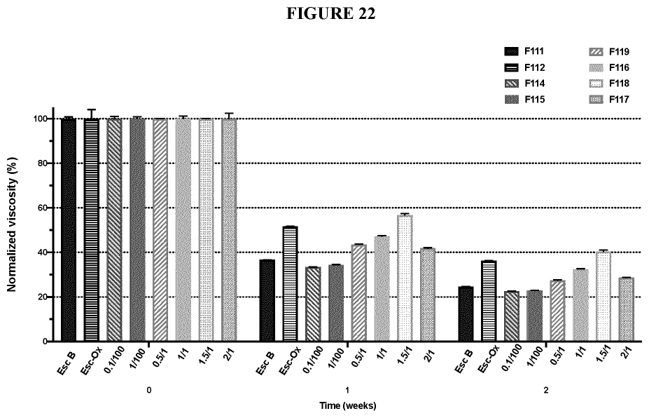

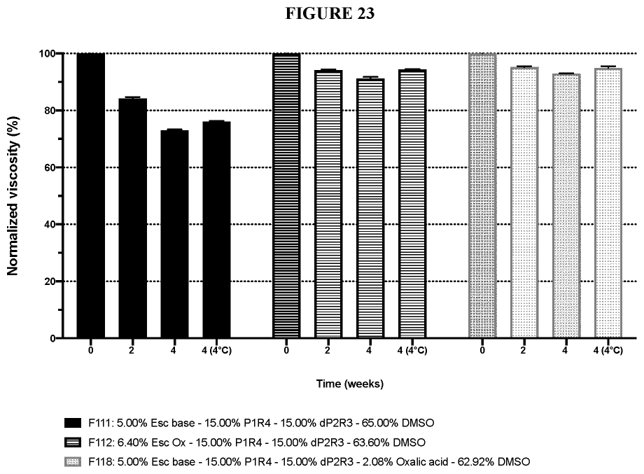

Whatever the tested conditions, drug recoveries remained stable with less than 5% deviation from the values measured at study start. For all formulations, a viscosity decrease was observed. Lowest viscosity decreases were obtained with Escitalopram oxalate control formulation and formulation with oxalic acid in a molar ratio of 1.5/1 with Escitalopram (F118). FIGS. 22 and 23 present results from the rheology analyses at 80° C. and RT respectively.

While the control escitalopram free base formulation F111 with no acid presents a strong viscosity decrease of around 25% its initial value after 4 weeks at RT or 4° C., in presence of oxalic acid, the degradation is highly reduced and is similar to the one of the escitalopram oxalate control formulation (F112).

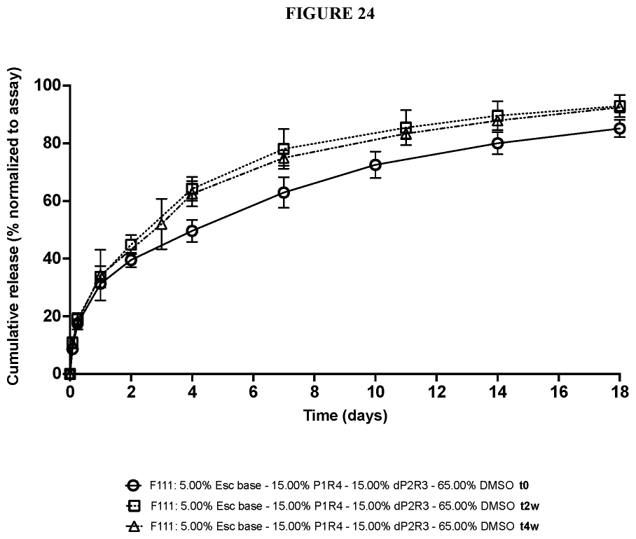

In vitro release profiles of F112 and F118 after 2 or 4 weeks of storage are similar to those obtained at study start. On the contrary, as shown on FIG. 24, the release profile of F111 is slightly accelerated with time, and present higher variability between replicates at early timepoints.

### Example 7: Degradation and Stability Studies of Atorvastatin Formulations

Forced degradation and stability studies were performed on atorvastatin calcium trihydrate formulations, as detailed in table 8 and according to example 3 with minor modifications. Drug contents were determined from samples dissolved in 40 mL of a 50/50 ACN/H₂O mixture and in vitro depots were formed in gelatin caspules size-00 before being transferred in a vial prefilled with 40 mL of PBS-1X+100 Tween 80.

TABLE 8

| Degradation study | | | Stability study | | |
|---|---|---|---|---|---|
| Timepoints | Temperature | Analyses | Timepoints | Temperature | Analyses |
| t0; t1w; t2w | 50° C. | Assay, visual | t0; t2w; t4w | RT | Assay, rheology, |
| | | observations | | | visual observations, |
| | | | | | IVR |

Table 9 discloses the compositions of tested atorvastatin formulations.

TABLE 9

| | | | | | | | | Oxalic | |
|---|---|---|---|---|---|---|---|---|---|
| Test | Atorvastatin % | P1R6 % | DB | DB % | s4P2R3 % | Solvent | Solvent % | acid % | Oxalic/Ator |
| Item | (w/w) | (w/w) | type | (w/w) | (w/w) | type | (w/w) | (w/w) | molar ratio |
| F125 | 19.60 | 10.00 | dP1R4 | 10.0 | — | DMSO | 60.40 | — | NA |
| F126 | 19.60 | 10.00 | dP1R4 | 10.0 | — | DMSO | 60.39 | 0.01 | 1/100 |
| F127 | 19.60 | 10.00 | dP1R4 | 10.0 | — | DMSO | 60.27 | 0.13 | 10/100 |
| F128 | 19.60 | 10.00 | dP1R4 | 10.0 | — | DMSO | 59.73 | 0.67 | 50/100 |
| F129 | 19.60 | 10.00 | dP1R4 | 10.0 | — | DMSO | 59.06 | 1.34 | 1/1 |
| F130 | 19.60 | 10.00 | dP1R4 | 10.0 | — | DMSO | 57.72 | 2.68 | 2/1 |
| F131 | 19.60 | — | — | — | 20.00 | DMSO | 60.40 | — | NA |
| F132 | 19.60 | 10.00 | dP1R4 | 10.0 | — | DMSO | 59.56 | 0.84 | 60/100 |
| F133 | 19.60 | 10.00 | dP1R4 | 10.0 | — | DMSO | 59.42 | 0.98 | 70/100 |
| F134 | 19.60 | 10.00 | dP1R4 | 10.0 | — | DMSO | 59.28 | 1.12 | 80/100 |
| F135 | 19.60 | 10.00 | dP1R4 | 10.0 | — | DMSO | 59.14 | 1.26 | 90/100 |
| F136 | 19.60 | — | — | — | 20.00 | DMSO | 59.28 | 1.12 | 80/100 |
| F137 | 19.60 | — | dP2R3 | 20.0 | — | DMSO | 60.40 | — | NA |
| F138 | 19.60 | — | dP2R3 | 20.0 | — | DMSO | 59.28 | 1.12 | 80/100 |
| F139 | 19.60 | 10.00 | dP1R4 | 10.0 | — | NMP | 60.40 | — | NA |
| F140 | 19.60 | 10.00 | dP1R4 | 10.0 | — | NMP | 59.28 | 1.12 | 80/100 |
| F141 | 9.80 | 10.00 | dP1R4 | 10.0 | — | DMSO | 70.20 | — | NA |
| F142 | 9.80 | 10.00 | dP1R4 | 10.0 | — | DMSO | 69.57 | 0.63 | 90/100 |
| F143 | 19.60 | — | — | — | — | DMSO | 80.40 | — | NA |
| F144 | 19.60 | — | — | — | — | DMSO | 79.28 | 1.12 | 80/100 |

NA: Not applicable

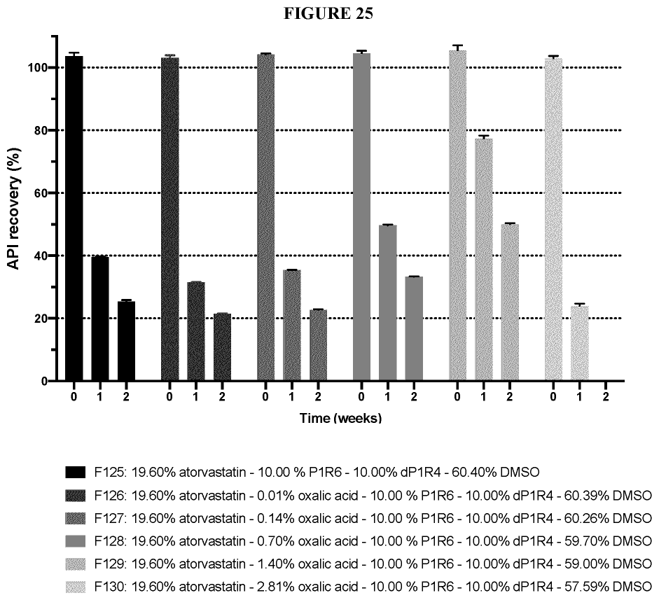

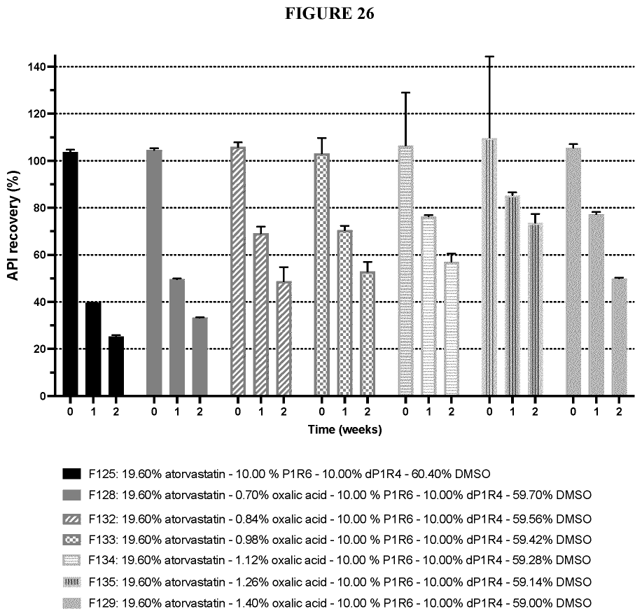

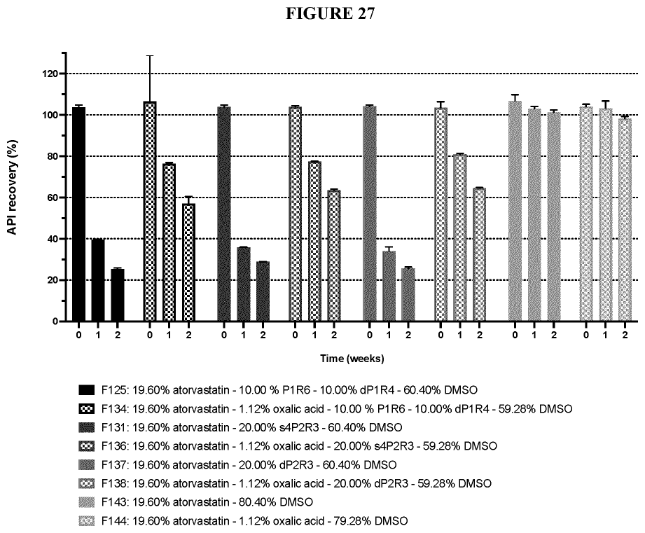

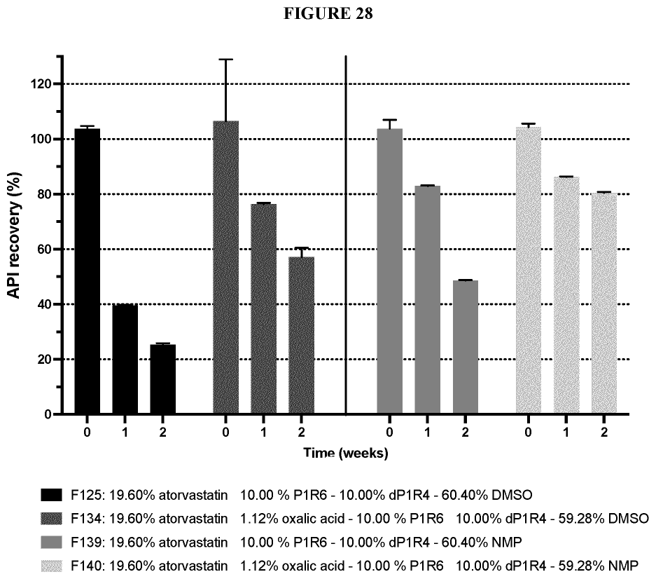

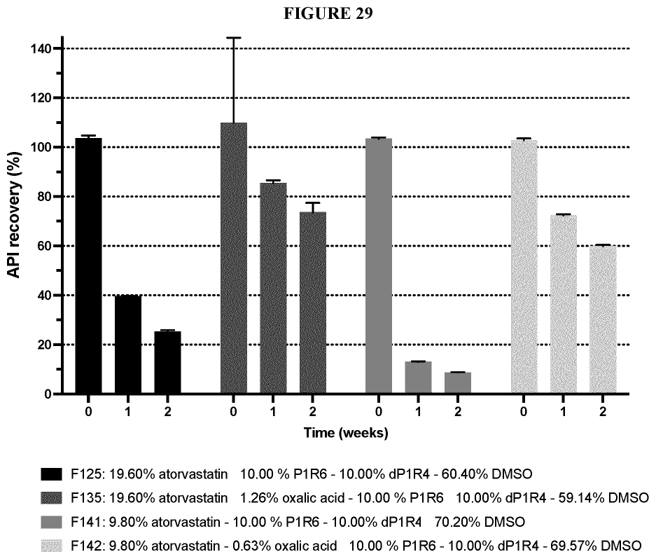

FIGS. 25 to 29 present results obtained from the 2-week forced degradation at 50° C. It can be observed that the addition of oxalic acid in between a 50/100 and up to a 100/100 oxalic/atorvastatin molar ratio, increases the API recovery with time. While the PEG-PLA copolymer type and/or structure had no impact on the degradation level, the solvent type as well as the initial API content led to different recovery levels.

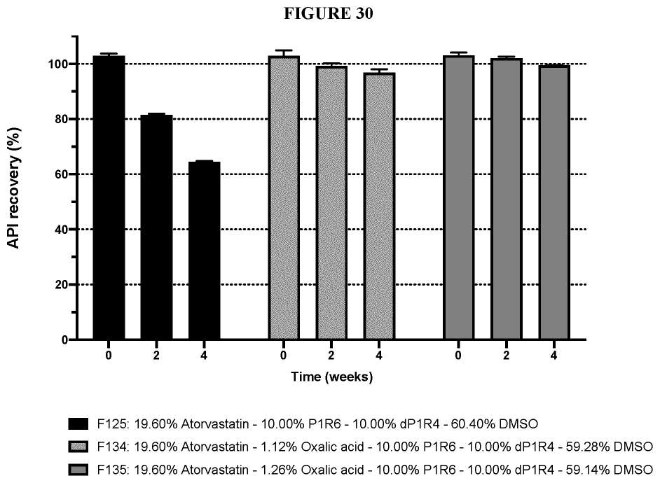

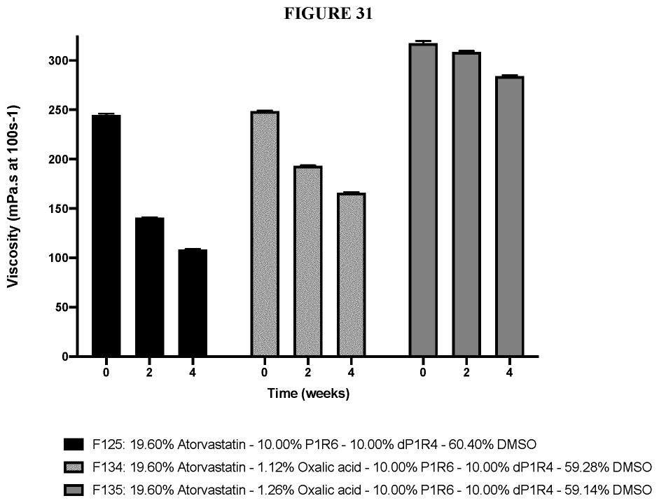

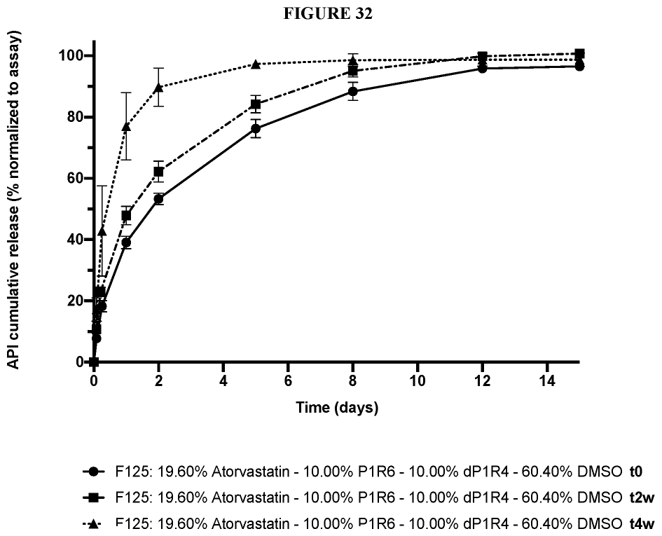

FIGS. 30 to 32 present results obtained from the 4-week stability study at RT. A clear improvement in formulation stability is observed in presence of oxalic acid. A difference of only 0.14% oxalic acid also had an impact, with higher drug recovery and lower viscosity decrease measured in the formulation containing more oxalic acid. In vitro release profiles of formulations F134 and F135 containing oxalic acid were similar over time. On the contrary, as illustrated on FIG. 32, the degradation of control formulation F125 led to an acceleration of the release of remaining API after 2 or 4 weeks of storage.

### Example 8: Pharmacokinetics (PK) Study of Octreotide Acetate Formulations

Selected octreotide acetate formulations were tested in a pharmacokinetics study in male adult rats. Drug products containing 2 mg of octreotide were subcutaneously administered in the interscapular area of the rats using 1 mL Soft Ject syringes and 23 G (1″ 0.6×25 mm) Terumo® needles. Injected formulation volumes were fixed to 90 μL. Blood samples were collected into EDTA tubes before injection and at different time points: 0.5 h, 1 h, 3 h, 8 h, 24 h, 48 h, 96 h, 168 h, 240 h, 336 h, 504 h and 672 h post dose. Blood samples were centrifuged and the plasma from each time point was retained. The plasma samples were analysed by LC/MS/MS for quantifying API content.

Table 10 discloses formulations compositions.

TABLE 10

| | API % | P1R6 % | dP2R3 % | DMSO % | | Excipient % |
|---|---|---|---|---|---|---|
| Formulation | (w/w) | (w/w) | (w/w) | (w/w) | Excipient | (w/w) |
| F162 | 2.2 | 11.0 | 33.0 | 53.3 | CaCl₂ | 0.5 |
| F165 | 2.2 | 10.9 | 32.8 | 53.2 | Pamoic acid | 0.8 |

Calculated PK parameters are detailed in table 11.

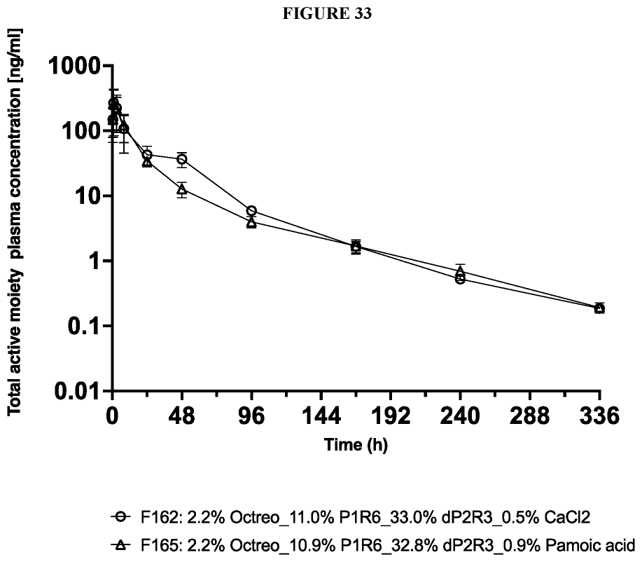

FIG. 33 illustrates the release profiles obtained in vivo. Data indicates that similar profiles are obtained from formulations containing pamoic acid or CaCl₂), with the two curves overlapping for most timepoints.

TABLE 11

| PK | Animal/ | tₘₐₓ⁽¹⁾ | Cₘₐₓ⁽²⁾ | AUC₀-t_{Dlast}^{(2) (3)} |
|---|---|---|---|---|
| parameters | group | (h) | (ng/mL) | (ng · h/mL) |
| F162 | 4 | 1.5 | 280 | 5034 |
| F165 | 4 | 1.5 | 281 | 4003 |

### Example 9: PK and Local Toxicity Studies of Octreotide Acetate Formulations

A second pharmacokinetics study of 10 days was performed on male adult rats with formulations F122 and F123 (see detailed composition in example 3). Drug products containing around 4.5 mg of octreotide were subcutaneously administered in the interscapular area of the rats using 1 mL Soft Ject syringes and 23 G (⅝″ 0.6×16 mm) Terumo® needles. Injected formulation volumes were fixed to 100 μL. Blood samples were collected into EDTA tubes before injection and at different time points: 0.5 h, 1 h, 3 h, 8 h, 24 h, 48 h, 96 h, 168 h and 240 h post dose. Two animals of each group were sacrificed 3 days after injection, and an extra blood collection at 72 h (D3) was performed prior euthanasia. Blood samples were centrifuged and the plasma from each time point was retained. The plasma samples were analyzed by LC/MS/MS for quantifying API content.

After euthanasia, injection sites were excised and fixed with formalin. Sections of the explants were stained with hematoxylin and eosin and a histopathological analysis was carried through microscopic observation by expert physiopathologists.

No significative differences were observed on histopathology analyses between the control formulation F123 and the formulation F122 containing oxalic acid, suggesting the good tolerability of the amount of acid used in the formulation.

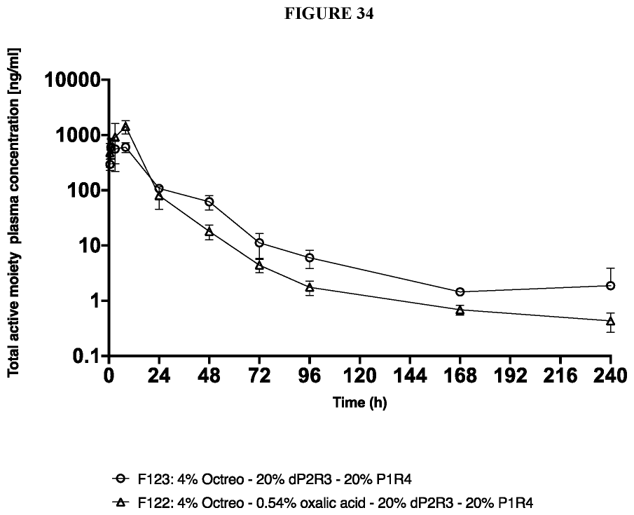

FIG. 34 illustrates the release profiles obtained in vivo. Data indicates that controlled releases are obtained from both formulations, and that the presence of oxalic acid within the formulation induced a higher initial burst, followed by a lower release level.

1. A pharmaceutical composition comprising or consisting of
a) at least one polyether-polyester copolymer, wherein the copolymer has the formula:
B(A)ₙ
wherein B represents a polyether and comprises polyethylene glycol (PEG), each A represents a polyester arm and n is an integer from 1 to 8;
b) at least one nucleophilic compound;
c) at least one organic solvent; and
d) up to 10% (w/w) of at least one acidic compound having a pKₐ(H₂O) of less than 3.

2. A pharmaceutical composition according to claim 1 wherein the at least one polyether-polyester copolymer a) is selected from;
i. a multi-arm copolymer having 3 to 8 polyester arms attached to a central core which is a multi-arm polyether comprising PEG and wherein each polyether arm has from 2 to 150 ethylene oxide repeat units and each polyester arm has from 4 to 200 repeat units; and
ii. a triblock copolymer, wherein the triblock copolymer has the formula:
Av-Bw-Ax
wherein A is a polyester and B is PEG and v and x are the number of repeat units ranging from 1 to 3,000 and w is the number of repeat units ranging from 3 to 300 and v=x or v≠x; and
iii. a diblock copolymer, wherein the diblock copolymer has the formula:
Cy-Az
wherein A is a polyester and C is an end-capped PEG and y and z are the number of repeat units with y ranging from 2 to 250 and z ranging from 1 to 3,000;
iv. or any combination thereof.

3. A pharmaceutical composition according to claim 1 or claim 2 wherein the at least one acidic compound each has a pKₐ(H₂O) of from −15.00 to 2.97, optionally from about −3.00 to about 2.90, optionally from about 0.50 to about 2.75, optionally from about 1.40 to about 2.75.

4. A pharmaceutical composition according to any preceding claim, wherein the composition is liquid at room temperature and forms a semi solid or solid implant when injected into an aqueous environment.

5. A pharmaceutical composition according to any preceding claim wherein the acidic compound d) is an inorganic acid or a carboxylic acid, optionally a polycarboxylic acid, optionally a di or tricarboxylic acid.

6. A pharmaceutical composition according to any preceding claim wherein the acidic compound d) is selected from aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid, tartaric acid citraconic acid, methylphosphonic acid, ethylphosphonic acid, propylphosphonic acid, butylphosphonic acid, pentylphosphonic acid, hexylphosphonic acid, heptylphosphonic, octylphosphonic acid, nicotinic acid, hydroiodic acid, chromic acid, trifluoromethane sulfonic acid, trichloroacetic acid, dichloroacetic acid, bromoacetic acid, chloroacetic acid, cyanoacetic acid, 2-chloropropanoic acid, 2-chlorobutanoic acid, 4-cyanobutanoic acid, perchloric acid, a phosphoric acid or a combination thereof.

7. A pharmaceutical composition according to any preceding claim wherein the acidic compound d) is selected from aspartic acid, benzene sulfonic acid, gentisic acid, dihydroxyfumaric acid, hydrochloric acid, hydrobromic acid, maleic acid, malonic acid, methanesulfonic acid, nitric acid, oxalic acid, oxaloacetic acid, pamoic acid, phosphoric acid, phtalic acid, pyruvic acid, sulfonic acid, sulfuric acid or tartaric acid or a combination thereof, preferably salicyclic acid, oxalic acid, malonic acid, sulfamic acid, pamoic acid or any combination thereof.

8. A pharmaceutical composition according to any preceding claim, wherein the polyester of the polyether-polyester copolymer a) is poly(D,L-lactic acid) (PLA), poly(D,L-lactic-co-glycolic acid) (PLGA) or poly(ε-caprolactone-co-lactic acid) (PCLA).

9. A pharmaceutical composition according to any of claim 2 to 8 wherein the end-capped polyethylene glycol is methoxy-polyethylene glycol.

10. A pharmaceutical composition according to any preceding claim wherein the polyester of the polyether-polyester copolymer a) is poly(D,L-lactic acid) (PLA).

11. A pharmaceutical composition according to any preceding claim wherein the polyether-polyester copolymer a) is a multi-arm copolymer i) having a molar ratio of the ester repeat unit to the ethylene oxide repeat unit of from 1 to 10, preferably from 2 to 6.

12. A pharmaceutical composition according to any preceding claim, wherein if the polyether-polyester copolymer a) is a multi-arm copolymer i) the central core is a multi-arm polyether which is obtainable from PEG and a polyol.

13. A composition according to claim 12 wherein the polyol comprises at least three hydroxyl groups, optionally wherein the polyol is a hydrocarbon substituted with at least three hydroxyl groups, optionally 3, 4, 5, 6 or 8 hydroxyl groups.

14. A composition according to claim 12 or claim 13 wherein the polyol is pentaerythritol (PE), dipentaerythritol, trimethylolpropane (TMP), glycerol, erythritol, xylitol, di(trimethylolpropane (diTMP) sorbitol, or inositol.

15. The composition according to any of claims 12 to 14 wherein the polyol further comprises one or more ether groups.

16. A pharmaceutical composition according to any of claims 2 to 10, wherein the at least one polyether-polyester copolymer a) is a mixture of a triblock copolymer ii) and a diblock copolymer iii).

17. A pharmaceutical composition according to any of claims 2 to 10 and 16 wherein the molar ratio of the ester repeat unit to the ethylene oxide repeat unit for the triblock copolymer ii) is from 0.5 to 22, preferably from 0.5 to 10, most preferably from 1 to 6.

18. A pharmaceutical composition according to any of claims 2 to 10 and 15 or 16 wherein the molar ratio of the ester repeat unit to the ethylene oxide repeat unit for the diblock copolymer iii) is from 0.8 to 15, preferably from 1 to 10.

19. A pharmaceutical composition according to any preceding claim, wherein the nucleophilic compound b) comprises one or more functional groups selected from —SH, —OH, a primary amine, a secondary amine, a tertiary amine, and combinations thereof.

20. A pharmaceutical composition according to any preceding claim, wherein the nucleophilic compound b) is an active pharmaceutical ingredient.

21. A pharmaceutical composition according to claim 20 wherein the active pharmaceutical ingredient is a free base or is a salt of an acid having a pKa(H₂O) of greater than 3.

22. A pharmaceutical composition according to claim 20 or claim 21 wherein the active pharmaceutical ingredient is octreotide acetate, liothyronine, escitalopram free base, atorvastatin calcium trihydrate or combination thereof.

23. A pharmaceutical composition according to any of claims 1 to 19 wherein the nucleophilic compound is not an active pharmaceutical ingredient and wherein the composition further comprises at least one active pharmaceutical ingredient.

24. A pharmaceutical composition according to claim 23 wherein the nucleophilic compound b) is an alcohol, optionally a C₁ to C₈ alcohol, optionally glycerol, sorbitol, methanol, ethanol, propanediol, propylene glycol, polyethylene glycol, preferably methanol, propylene glycol, polyethylene glycol or derivatives or mixtures thereof.

25. A pharmaceutical composition according to claim 23 wherein the nucleophilic compound b) is a saccharide, disaccharide or polysaccharide, optionally sucrose, dextrose, cyclodextrin, chitosan or mixtures thereof.

26. A pharmaceutical composition according to claim 23 wherein the nucleophilic compound b) is an amino acid, peptide, or polypeptide, optionally lysine, arginine, histidine or serine.

27. A pharmaceutical composition according to claim 23 wherein the nucleophilic compound b) is water.

28. A pharmaceutical composition according to claim 23 wherein the nucleophilic compound b) is a further organic solvent, optionally pyrrolidone-2, glycofurol, pyridine, nitromethane, triethylamine, N,N-dimethylaniline, N,N-diemthyldecanamide, N,N-dimethyloctanamide, 2,4,6-collidine or mixtures thereof.

29. A pharmaceutical composition according to claim 23 to 28 wherein the nucleophilic compound b) is a solubility enhancer, a porogen or a phase exchange modifier.

30. A pharmaceutical composition according to any preceding claim, wherein the at least one organic solvent c) is selected from the group consisting of benzyl alcohol, benzyl benzoate, dimethyl isosorbide (DMI), dimethyl sulfoxide (DMSO), ethyl acetate, ethyl benzoate, ethyl lactate, glycerol formal, methyl ethyl ketone, methyl isobutyl ketone, N-ethyl-2-pyrrolidone, N-methyl-2-pyrrolidinone (NMP), pyrrolidone-2, triacetin, tributyrin, tripropionin, glycofurol or a mixture thereof, preferably DMSO, NMP and mixtures thereof.

31. A pharmaceutical composition according to any preceding claim, wherein the acidic compound d) has a pKₐ(DMSO) lower than 10, preferably lower than 8.

32. A pharmaceutical composition according to any preceding claim, wherein the amount of the at least one acidic compound d) is from 0.005% (w/w) to 10% (w/w), optionally 0.55% (w/w) to 10% (w/w), or 0.005% (w/w) to 0.45% (w/w), preferably 0.01% (w/w) to 4.0% (w/w) of the total composition.

33. A pharmaceutical composition according to any of preceding claim, wherein the molar amount of the acidic compound d) is 0.05% to 300% relative to the molar amount of the nucleophilic compound b), preferably 0.1% to 250%.

34. A pharmaceutical composition according to any preceding claim, wherein the nucleophilic compound b) contains at least one —OH group and wherein the molar amount of the acidic compound d) is equal to or lower than 100% relative to the molar amount of the nucleophilic compound, preferably 0.05% to 100% relative to the molar amount of the nucleophilic compound.

35. A pharmaceutical composition according to any preceding claim, wherein the nucleophilic compound b) contains at least one nitrogen containing reactive group such as a primary or secondary amine, and wherein the molar amount of the acidic compound d) is equal to or greater than 100% relative to the molar amount of the nucleophilic compound, preferably 100% to 300% relative to the amount of the nucleophilic compound.

36. A pharmaceutical composition according to any preceding claim, wherein the total amount of the polyether-polyester copolymer a) is 2% (w/w) to 80% (w/w), optionally 10 to 50% (w/w), optionally 20 to 40% (w/w) of the total composition.

37. A pharmaceutical composition according to any of claims 2 to 15 or 19 to 36, wherein the polyether-polyester copolymer a) is a multi-arm copolymer i) and the amount of the multi-arm copolymer is from 20 to 60% (w/w), optionally 20 to 50% (w/w) of the total composition.

38. A pharmaceutical composition according to any of claims 2 to 10 or 16 to 36, wherein the amount of the diblock copolymer is from 2 to 30% (w/w), optionally 10 to 30% (w/w), optionally 10 to 20% (w/w) of the total composition; and the amount of the triblock copolymer is from 2 to 30% (w/w), optionally 10 to 30% (w/w), optionally 10 to 20% (w/w) of the total composition.

39. A pharmaceutical composition according to any of claims 20 to 38, wherein the amount of the active pharmaceutical ingredient is 0.05% (w/w) to 60% (w/w), optionally 0.05 to 20% (w/w), optionally 0.05 to 10% (w/w), optionally 0.05 to 5% (w/w), optionally 0.05 to 2% (w/w) of the total composition.

40. A pharmaceutical composition according to any preceding claim, wherein the amount of the organic solvent is at least 20% (w/w) of the total composition, optionally 20 to 80% (w/w), optionally 20 to 60% (w/w).

41. A pharmaceutical composition according to any preceding claim, wherein the composition is stable for at least 2 weeks storage at room temperature or 2 to 8° C., preferably at least 4 weeks storage at room temperature or 2 to 8° C.

42. A pharmaceutical composition according to any preceding claim wherein the concentration of the active pharmaceutical ingredient in the composition reduces by less than 20%, preferably less than 10%, more preferably less than 5% after 2 weeks storage at room temperature or 2 to 8° C., preferably 4 weeks storage at room temperature or 2 to 8° C. relative to the initially formulated composition.

43. A pharmaceutical composition according to any preceding claim wherein the dynamic viscosity of the composition reduces by less than 10%, preferably less than 5% after 2 weeks storage at room temperature or 2 to 8° C., preferably 4 weeks storage at room temperature or 2 to 8° C. relative to the initially formulated composition.

44. A method for preparing a pharmaceutical composition as described in any preceding claim comprising or consisting of the steps of:
i. dissolving the at least one polyether-polyester copolymer a) as defined in any preceding claim in the at least one organic solvent c);
ii. adding to the product of step i) at least one acidic compound d) as defined in any preceding claim and at least one nucleophilic compound b) as defined in any preceding claim, optionally wherein the nucleophilic compound b) is an active pharmaceutical ingredient; and
iii. homogenizing the product of step ii), thereby obtaining the pharmaceutical composition.

45. A method according to claim 44 wherein the at least one acidic compound and the at least one nucleophilic compound do not form a salt or complex prior to step ii).

46. A method according to claim 44 or claim 45 wherein the at least one acidic compound and the at least one nucleophilic compound are not contacted or mixed together prior to step ii).

47. A method according to any of claims 44 to 46 wherein step ii) consists of mixing the components in a single step.

48. A method for preparing a pharmaceutical composition as described in any of claims 1 to 43 comprising or consisting of the steps of:
i. dissolving the at least one polyether-polyester copolymer a) as defined in any preceding claim in the at least one organic solvent c);
ii. adding to the product of step i) at least one acidic compound d) as defined in any preceding claim or at least one nucleophilic compound b) as defined in any preceding claim, and then homogenizing the product;
iii. if at least one acidic compound d) is added in step ii) then subsequently adding at least one nucleophilic compound b) as defined in any preceding claim; or if at least one nucleophilic compound b) is added in step ii) then subsequently adding at least one acidic compound d) as defined in any preceding claim; and
iv. homogenizing the product of step iii), thereby obtaining the pharmaceutical composition; optionally wherein the nucleophilic compound b) is an active pharmaceutical ingredient.

49. A method according to any of claims 44 to 48, wherein the nucleophilic compound is not an active pharmaceutical ingredient and an active pharmaceutical ingredient is added after step i).

50. A method according to any of claims 44 to 49, wherein the active pharmaceutical ingredient is previously dissolved in the organic solvent c).

51. A method according to any of claims 44 to 50, wherein the acidic compound d) is previously dissolved in the organic solvent c).

52. A method according to any of claims 44 to 51, wherein the nucleophilic compound b) is previously dissolved in the organic solvent c).

53. A method according to any of claim 44 to 52 wherein the pharmaceutical composition obtained in step iii. or iv. is filtered.

54. A pharmaceutical composition obtainable or obtained by the method of any of claims 44 to 53.
